(19) United States
(12) Patent Application Publication
(10) Pub. No.: US 2026/0232315 A1
(43) Pub. Date: Aug. 13, 2026

(54) POWERED SAFETY CHECKS FOR SURGICAL DEVICES AND ASSOCIATED SYSTEMS AND METHODS

(71) Applicant: Lexington Medical, Inc., Bedford, MA (US)

(72) Inventors: Andrew Marecki, Wilbraham, MA (US); Jameson Kief, Wakefield, MA (US)

(21) Appl. No.: 19/052,239

(22) Filed: Feb. 12, 2025

(51) Int. Cl. A61B 17/072; A61B 17/00; G16H 40/63

(52) U.S. Cl. CPC A61B 17/072; G16H 40/63; A61B 2017/00039; A61B 2017/00398; A61B 2017/00469; A61B 2017/07271

(57) ABSTRACT

Safety checks for surgical devices (and associated systems, devices, and methods) are disclosed herein. For example, some embodiments of the present technology are directed to one or more safety checks that can detect hardware and/or software failures associated with a surgical device. The safety checks can be automatically performed at the beginning of a new power cycle. Example safety checks can include data integrity checks, voltage checks, motor encoder functionality checks, reload checks, and error code checks. If the safety check determines that the surgical device is suitable for performing a medical procedure, the system can notify the physician of the same. If the safety check determines that the surgical device is not suitable for performing a medical procedure, the system can likewise notify the physician of the same.

### TECHNICAL FIELD

The present technology relates generally to safety checks, such as for surgical devices. For example, several embodiments of the present technology relate to powered safety checks for motor-driven surgical stapler devices and associated systems and methods.

### BACKGROUND

A surgical handle assembly and/or a surgical reloadable cartridge assembly can be used in a number of surgical devices. One example includes use in—or as part of—a surgical stapler. A surgical stapler is a fastening device used to clamp tissue between opposing jaw structures to join tissue using surgical fasteners. Surgical staplers can include two elongated members used to clamp the tissue. One of the elongated members can include one or more staple cartridges, and the other elongated member can include an anvil that can be used to form a staple when driven from the staple cartridge. Some surgical staplers are equipped with an electric motor that can provide the power to clamp tissue, deliver staples, and provide power for other aspects of a surgical stapler.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments shown, but are provided for explanation and understanding.

FIG. 1 is a block diagram of a surgical system configured in accordance with various embodiments of the present technology.

FIG. 2 is a partially schematic diagram of a surgical stapling apparatus configured in accordance with various embodiments of the present technology.

FIG. 3 is a partially schematic, partial exploded view of a surgical handle assembly configured in accordance with various embodiments of the present technology.

FIGS. 4A and 4B are partially schematic top and bottom diagrams, respectively, of a circuit board configured in accordance with various embodiments of the present technology.

FIG. 5 is a flowchart illustrating a method for performing a handle apparatus data integrity check in accordance with various embodiments of the present technology.

FIG. 6 is a flowchart illustrating a method for performing a control unit data integrity check in accordance with various embodiments of the present technology.

FIG. 7 is a flowchart illustrating a method for performing a software/firmware check between a handle apparatus and a control unit in accordance with various embodiments of the present technology.

FIG. 8 is a flowchart illustrating a method for performing a handshake protocol between a handle apparatus and a control unit in accordance with various embodiments of the present technology.

FIG. 9 is a flowchart illustrating a method for performing a handle apparatus voltage input check in accordance with various embodiments of the present technology.

FIG. 10 is a flowchart illustrating a method for performing a voltage input check in accordance with various embodiments of the present technology.

FIG. 11 is a flowchart illustrating a method for performing a control unit output power check in accordance with various embodiments of the present technology.

FIG. 12 is a flowchart illustrating a method for performing a motor encoder functionality check in accordance with various embodiments of the present technology.

FIG. 13 is a flowchart illustrating a method for performing a reload assembly check in accordance with various embodiments of the present technology.

FIG. 14 is a flowchart illustrating a method for performing a handle apparatus error code check in accordance with various embodiments of the present technology.

FIG. 15 is a flowchart illustrating a method for performing a control unit error code check in accordance with various embodiments of the present technology.

FIG. 16 is a flowchart illustrating a method 1600 for communicating a system operating state in accordance with various embodiments of the present technology.

### DETAILED DESCRIPTION

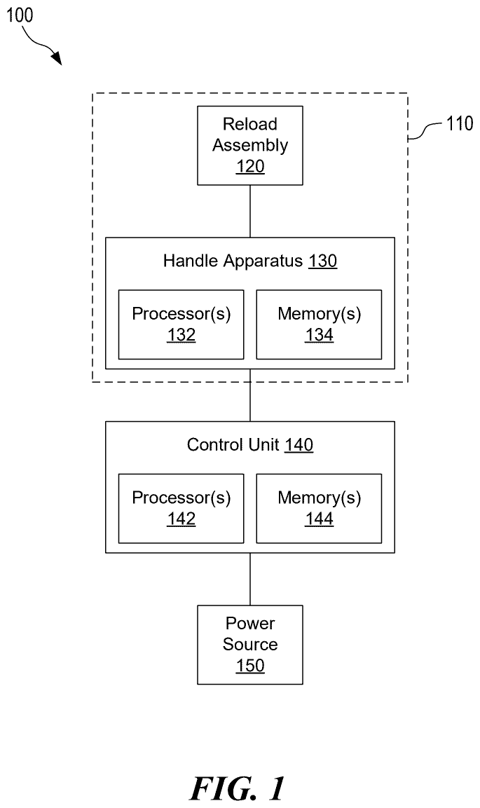

The present disclosure is generally directed to safety checks for surgical devices and associated systems and methods. Specific details of several embodiments of the present technology are described herein with reference to FIGS. 1-16. Although many of the embodiments are described below with reference to safety checks for use with motor-driven surgical stapler devices, other applications and other embodiments in addition to those described herein are within the scope of the present technology. For example, unless otherwise specified or made clear from context, the safety checks of the present technology can be used with other devices, such as surgical handle assemblies for other non-stapler medical devices.

It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the term “physician” shall be understood to include any type of medical personnel who may be performing or assisting a medical procedure and, thus, is inclusive of a doctor, a nurse, a clinician, a medical technician, other similar personnel, and any combination thereof. As used herein, the term “patient” and “subject” should be considered to include human and/or non-human (e.g., animal) patients upon which a medical procedure is being performed.

### A. Overview

Physicians often use surgical devices to perform various medical procedures on patients. For example, physicians can use surgical staplers to clamp tissue between opposing jaw structures and join tissue using surgical fasteners (e.g., staples, sutures). With the increasing complexity of such surgical devices, the number of potential hardware and software failures can be significant. For example, surgical devices can include a modular assembly of multiple swappable components, and one or more components unsuitable/inappropriate for a particular procedure may be attached. As another example, surgical devices can routinely receive software updates, and such updates may include corrupt data. These and other hardware/software failures can not only result in incorrect use of and damage to the surgical devices, but also pose safety risks to the patient.

To address the above concerns, several embodiments of the present technology are directed to one or more safety checks that can detect hardware and/or software failures associated with a surgical device (e.g., a non-robotic, handheld surgical device) prior to use (e.g., at the beginning of a new power cycle), and notify a physician of the same. For example, the safety checks can include memory/data integrity checks, voltage checks, motor encoder functionality checks, reload assembly presence checks, and error code checks.

### B. Selected Embodiments of Surgical Systems and Associated Devices and Methods

### 1. Surgical Systems

FIG. 1 is a block diagram of a surgical system 100 (“the system 100”) configured in accordance with various embodiments of the present technology. The system 100 can include a surgical device 110, a control unit 140, and a power source 150. In the illustrated embodiment, the surgical device 110 includes a reload assembly 120 and a handle apparatus 130 operably coupled to the reload assembly 120. The various components of the system 100 can be operably coupled to one another via wired and/or wireless connections.

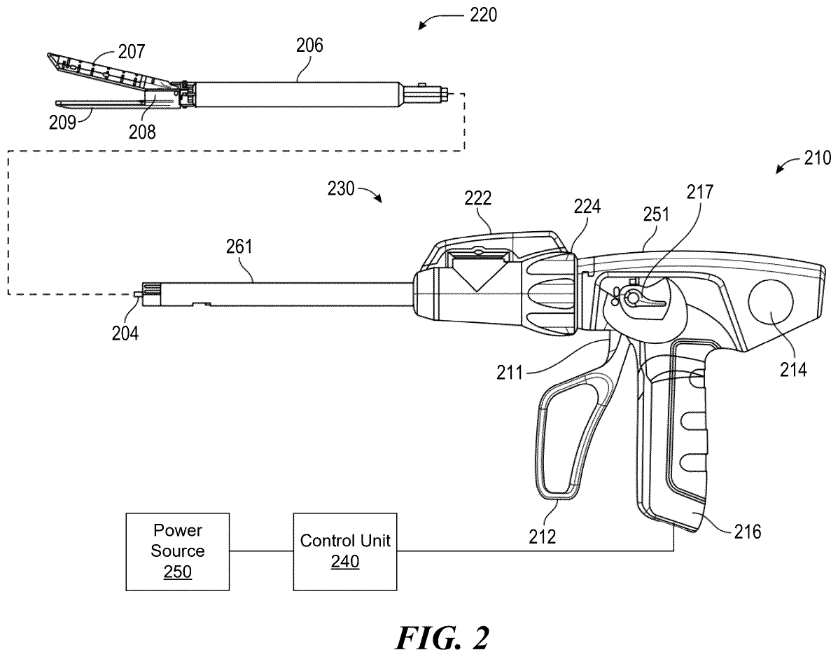

The surgical device 110 can include a non-robotic, handheld surgical device. For example, the surgical device 110 can comprise a surgical stapling apparatus, the reload assembly 120 can include jaws for clamping and stapling tissue, and the handle apparatus 130 can be used to control the reload assembly 120. An example surgical stapling apparatus is described in detail below with reference to FIGS. 2-6. In other examples, the surgical device 110 can include non-stapler medical devices.

The control unit 140 can include one or more inputs and one or more outputs (e.g., cable ports, communication module units). At least one of the outputs can be connected to the handle apparatus 130 for power transfer and/or data transfer. In these and other embodiments, at least one of the inputs can be connected to the handle apparatus 130 for data transfer. In some embodiments, the control unit 140 can be connected to other surgical devices or tools (e.g., an endoscope) and/or a display.

The power source 150 can include an energy storage component (e.g., a battery), an electrical outlet connected to an electrical grid, etc. In some embodiments, the system 100 omits the control unit 140 and/or the power source 150. In some embodiments, the handle apparatus 130 can be directly connected to the power source 150 (e.g., rather than through the control unit 140 as shown in FIG. 1). Additionally, or alternatively, the handle apparatus 130 can include an internal power source (e.g., a battery).

As shown in FIG. 1, the handle apparatus 130 can include one or more processors 132 and one or more memories 134, and the control unit 140 can include one or more processors 142 and one or more memories 144. Each of the processors 132 and 142 (e.g., CPU(s), GPU(s), HPU(s), etc.) can be (i) a single processing unit or (ii) multiple processing units co-located in a single device or distributed across multiple devices. The processors 132 and 142 can process various signals received from the control unit 140 or the handle apparatus 130, respectively, and/or other devices (e.g., an endoscope). Each of the memories 134 and 144 can include any suitable type of memory. For example, the memories 134 and/or 144 can include volatile and/or non-volatile memory devices. As more specific examples, the memories 134 and/or 144 can include random access memory (RAM), electrically erasable programmable read-only memory (EEPROM), magnetic disks or tapes, and/or flash memory.

In operation, as discussed in further detail herein, the processors 132 of the handle apparatus 130 and/or the processors 142 of the control unit 140 can perform one or more safety checks associated with the surgical device 110. For example, the processors 132 and/or 142 can process received signals, access data stored in the memories 134 and/or 144, and run various safety check algorithms, among other functions described in greater detail below.

### 2. Surgical Devices

FIG. 2 is a partially schematic diagram of a surgical stapling apparatus 210 configured in accordance with various embodiments of the present technology. The surgical stapling apparatus 210 can include a surgical reloadable cartridge assembly 220 (e.g., a disposable loading unit) and a surgical handle assembly 230 (e.g., a surgical stapling handle assembly). It is appreciated that the surgical stapling apparatus 210 can be an example of the surgical device 110 of FIG. 1, the surgical reloadable cartridge assembly 220 can be an example of the reload assembly 120 of FIG. 1, and the surgical handle assembly 230 can be an example of the handle apparatus 130 of FIG. 1. The surgical reloadable cartridge assembly 220 can be releasably secured to a distal end of an elongated body 261 of the surgical handle assembly 230, such as to a drive shaft 204 of the surgical handle assembly 230.

In the illustrated embodiment, the reloadable cartridge assembly 220 includes a shaft 206, a first elongated member 207, and a second elongated member 209. The first elongated member 207 and the second elongated member 209 can be used to clamp tissue, and are also referred to herein as “jaws.” One of the elongated members (e.g., the first elongated member 207) can house one or more staple cartridges. The other elongated member (e.g., the second elongated member 209) can include an anvil that can be used to form a staple when driven from the staple cartridge (e.g., by a blade assembly (not shown in FIG. 2)). In some embodiments, the reloadable cartridge assembly 220 can further include a stabilizing bracket 208 that can help to hold the first elongated member 207 and the second elongated member 209 in alignment with one another and/or that can function as a tissue shield to prevent or hinder tissue from being clamped between the first elongated member 207 and the second elongated member 209 at a location more proximal than a distal end of the stabilizing bracket 208. In these and other embodiments, the reloadable cartridge assembly 220 can include one or more rows of staples having a linear length. For example, a row of staples can have a linear length between approximately 30 mm and approximately 60 mm. In a number of embodiments, third party reloadable cartridges and/or reloadable cartridge assemblies may be used with the surgical handle assembly 230 and embodiments of the surgical handle assembly 230 may be configured to receive the same.

The surgical handle assembly 230 can include a radial positioner 224, an articulation assembly activated by an articulation knob 222, a non-movable handle 216 (“the stationary handle 216”), and a movable handle 212. When the reloadable cartridge assembly 220 is releasably secured to the distal end of the elongated body 261 of the surgical handle assembly 230, the reloadable cartridge assembly 220 can be actuated using the articulation knob 222 and/or the radial positioner 224 to reach a stapling site. For example, the radial positioner 224 can be used to rotate the reloadable cartridge assembly 220. Additionally, or alternatively, the articulation knob 222 can be used to position the first elongated member 207 and/or the second elongated member 209 at a particular angle for stapling. The articulation knob 222 can be rotationally actuatable, and the reloadable cartridge assembly 220 can rotate about an axis of a particular plane in response to the articulation knob 222 being rotationally actuated by a physician. The movable handle 212 can be used to clamp and unclamp the first elongated member 207 and the second elongated member 209 together (e.g., to clamp or grip tissue).

As shown, the surgical handle assembly 230 further includes a power trigger 211, a selector lever 217, a manual retraction or bailout handle 251, and a user feedback mechanism 214. Although not shown in FIG. 2, the surgical handle assembly 230 can include an internal power source, such as a battery. The internal power source (e.g., battery) can be rechargeable (e.g., via an AC power supply) or disposable. If a rechargeable battery is used, the battery can be positioned so that it can be either removed or recharged. If a disposable battery is used, the stationary handle 216 can include a drain so that the battery can be drained prior to disposal. In these and other embodiments, the surgical handle assembly 230 can be electrically coupled to a control unit 240 (e.g., the control unit 140 of FIG. 1 or another control unit configured in accordance with various embodiments of the present technology) and/or a power source 250 (e.g., the power source 150 of FIG. 1 or another power source configured in accordance with various embodiments of the present technology), such as an external power source. The power source 250 can supply AC or DC current to the surgical handle assembly 230 to power various electronics of the surgical handle assembly 230.

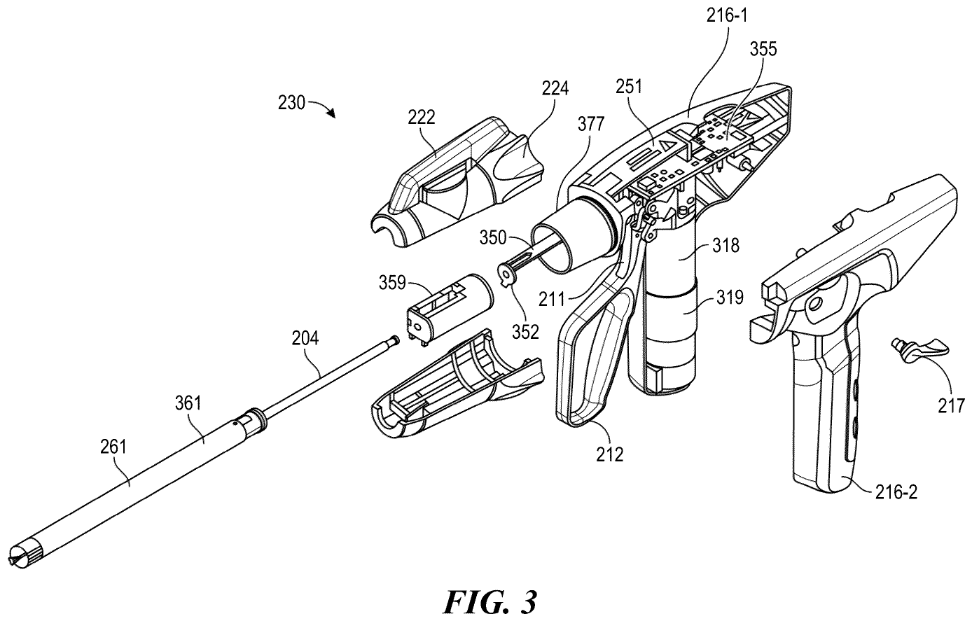

FIG. 3 is a partially schematic, partial exploded view of the surgical handle assembly 230 of FIG. 2. As discussed above, a distal portion of the surgical handle assembly 230 can include the articulation knob 222 and the radial positioner 224. The surgical handle assembly 230 can include a drive assembly usable to control and effect movement of the reloadable cartridge assembly. In the illustrated embodiment, the drive assembly includes the drive shaft 204 (also referred to herein as a “drive rod”) and a gear rack (not shown). The drive shaft 204 can cooperate with the gear rack to effect movement of a reloadable cartridge assembly (e.g., the reloadable cartridge assembly 220 of FIG. 2) at least when the reloadable cartridge assembly is attached to the surgical handle assembly 230.

FIG. 3 illustrates a first handle half 216-1 and a second handle half 216-2 that together provide the stationary handle 216 for a user of the surgical handle assembly 230 and that together house a drivetrain 318 and/or other electrical and mechanical mechanisms of the surgical handle assembly 230. The first handle half 216-1 and/or the second handle half 216-2 may also include components that are used in the clamping and/or unclamping of the jaws.

In some embodiments, the drivetrain 318 can include a number of gears (not shown), an internal power source (not shown), such as a battery, an electric motor 319, and/or one or more encoders (e.g., linear encoders, rotary encoders; not shown) operably coupled to the electric motor 319. The electric motor 319 can be battery-powered and/or can be connected to an external power source (e.g., the power source 250 of FIG. 2). In these and other embodiments, the drivetrain 318 can include the electric motor 319 and a number of gears, and a battery can be located elsewhere (e.g., another position between the first handle half 216-1 and the second handle half 216-2).

The power trigger 211 and the selector lever 217 can be used to activate the drivetrain 318. The movable handle 212 can be used to clamp and unclamp the first elongated member 207 (FIG. 2) and the second elongated member 209 (FIG. 2) together, and the selector lever 217 can allow power to flow to the electric motor and/or block the power trigger 211 from being activated. Also shown in FIG. 3 is an outer shaft 361 of the elongated body 261, a nose cone 377, the bailout handle 251, a circuit board 355, an articulation lock 359, toggle plates 352, and a bailout bar 350. The circuit board 355 can include a microcontroller or microprocessor.

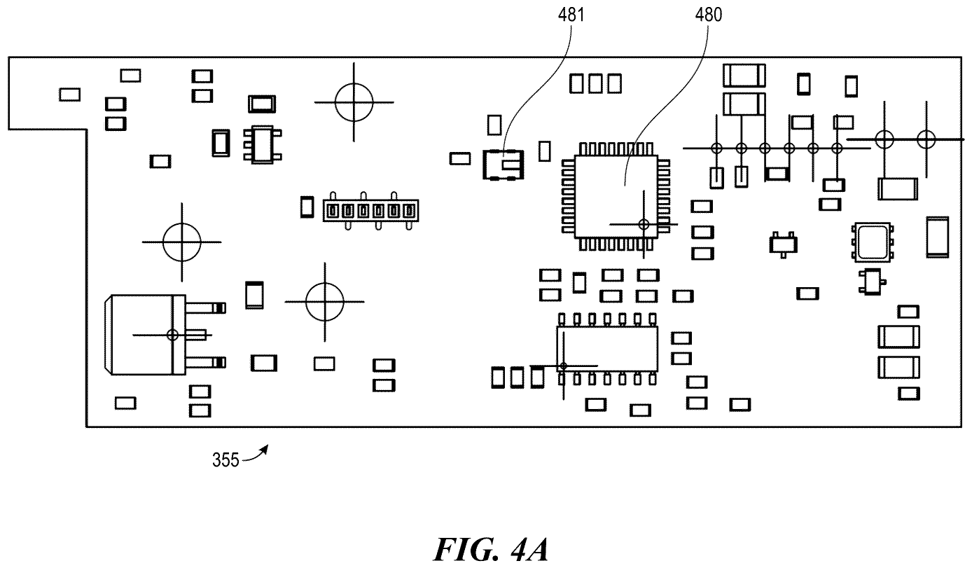

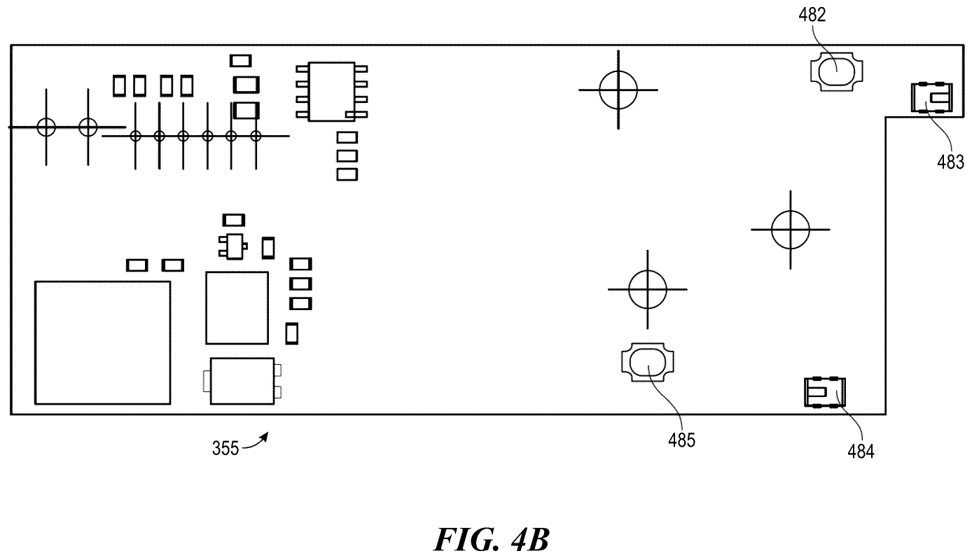

FIGS. 4A and 4B are partially schematic top and bottom diagrams, respectively, of the circuit board 355 of FIG. 3. In some embodiments, at least some aspects of the surgical handle assembly 230 of FIGS. 2 and 3 can be controlled via the circuit board 355. Referring to FIG. 4A, the circuit board 355 can include a microcontroller 480 (or microprocessor) for controlling various functions (e.g., all or a subset of one or more of the safety check algorithms disclosed herein) of the surgical handle assembly 230. The microcontroller 480 can be an example of the processors 132 of FIG. 1 or of other processors configured in accordance with various embodiments of the present technology. As discussed further herein, the microcontroller 480 can run through a series of safety checks upon start up when the surgical handle assembly 230 is turned on or, in some embodiments, when it is plugged in.

The circuit board 355 can also include various switches. For example, referring to FIG. 4A, the circuit board 355 can include a switch 481 that can interact with the bailout handle 251. More specifically, when the switch 481 is closed via contact with a ‘U’-shaped latch of the bailout handle 251, power can be supplied to the circuit board 355. On the other hand, when the bailout handle 251 is lifted such that the ‘U’-shaped latch is not in contact with the switch 481, the switch 481 can be open such that no power flows to the circuit board 355.

Referring now to FIG. 4B, the circuit board 355 can additionally, or alternatively, include switches 482-485. Switch 482 can be a power switch for the drivetrain 318 of FIG. 3. More specifically, when the power trigger 211 (FIGS. 2 and 3) is pulled, the power trigger 211 can press a protrusion of an intermediate lever into contact with the switch 482 on the circuit board 355, permitting power to flow to the drivetrain 318 and/or permitting the drivetrain 318 to move the gear rack distally. In some embodiments, the power switch 482 can be an open/close switch such that the drivetrain 318 receives relatively constant power when the power trigger 211 is pulled. In other embodiments, the power switch 482 can be a variable switch (e.g., a variable resistor, varistor, potentiometer, or another analog sensor) such the drivetrain 318 receives more power (and therefore runs faster) as the power trigger 211 is squeezed harder or actuated by a greater amount.

Switch 483 of the circuit board 355 can be a reload switch. When a reloadable cartridge assembly (e.g., the reloadable cartridge assembly 220 of FIG. 2) is attached to the surgical handle assembly 230 (FIG. 2), a cam can be pressed into the switch 483 to indicate the attachment. Thus, when no reloadable cartridge assembly is attached to the surgical handle assembly 230, the switch 483 can be open such that the surgical handle assembly 230 is prevented from being electrically activated.

Switch 484 of the circuit board 355 can be a ready-to-fire switch. For example, when the selector lever 217 (FIG. 2) is rotated to an unlocked position, a selector cam can rotate such that a protrusion engages the switch 484 on the circuit board 355. When the switch 484 of the circuit board 355 is activated, the surgical handle assembly 230 can be placed in a ready-to-fire state in which energy can be allowed to flow to the drivetrain 318 (e.g., in response to actuation of the power trigger 211 and/or to move the gear rack distally).

Switch 485 of the circuit board 355 can be a reverse switch. For example, when the selector lever 217 (FIG. 2) is rotated to a reverse position, a cam can rotate a reverse cam and cause a protrusion on the reverse cam to engage the switch 485 on the circuit board 355. In turn, the drivetrain 318 (FIG. 3) can be controlled to move in reverse such that the gear rack and the drive shaft 204 (FIGS. 2 and 3) of the surgical handle assembly 230 are retracted proximally.

Additional details regarding surgical stapling apparatuses, surgical handle assemblies, and, in particular, motorized surgical handle assemblies, are provided (1) in U.S. patent application Ser. No. 17/833,302, titled “MOTORIZED SURGICAL HANDLE ASSEMBLY, and filed on Jun. 6, 2022, and (2) in U.S. patent application Ser. No. 18/391,251, titled “SAFETY CONTROL ALGORITHMS FOR SURGICAL DEVICES, INCLUDING SAFETY CONTROL ALGORITHMS FOR SURGICAL STAPLER DEVICES, AND ASSOCIATED SYSTEMS, DEVICES, AND METHODS,” and filed on Dec. 20, 2023, the disclosures of which are incorporated by reference herein in their entireties.

It is appreciated that while select features of the present technology may be described herein with reference to the surgical stapling apparatus 210 illustrated in FIGS. 2-4B, safety checks configured in accordance with various embodiments of the present technology can be used with and for other surgical and/or medical devices, including non-stapling surgical and/or medical devices.

### 3. Associated Safety Check Methods

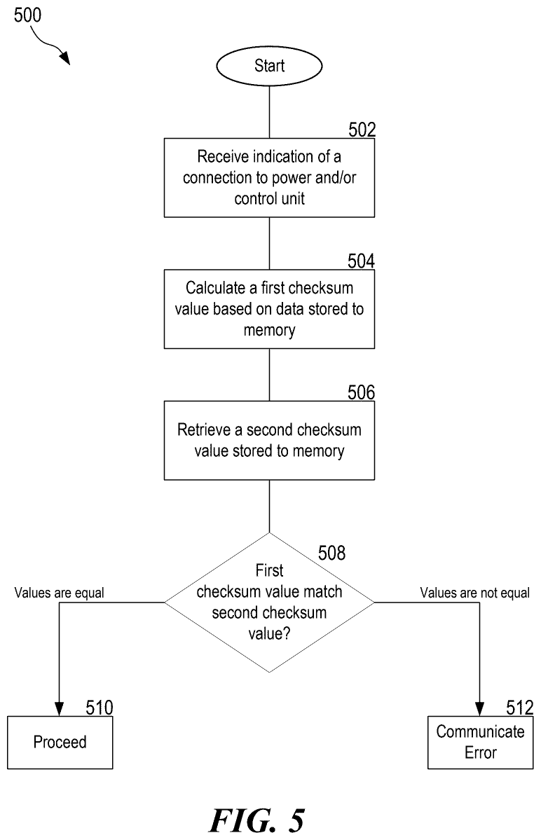

FIGS. 5-16, described in detail below, are flowcharts illustrating various safety check methods or algorithms in accordance with various embodiments of the present technology. In some embodiments, the safety checks described herein are performed by one or more processors (e.g., the processors 142 of the control unit 140, the processors 132 of the handle apparatus 130, and/or the microcontroller 480 of the surgical handle assembly 230) based on instructions stored on one or more memories (e.g., the memory 144 of the control unit 140 and/or the memory 134 of the handle apparatus 130) or other non-transitory computer-readable medium. All or a subset of the various safety check methods described herein can be performed sequentially and/or simultaneously. In some embodiments, one or more of the safety checks methods described herein are performed at the beginning of a new power cycle (e.g., when the surgical device is connected (e.g., initially connected) to a control unit and/or power supply, when the surgical device and/or the control unit is powered on or otherwise connected to power).

It will be appreciated that the safety checks illustrated in FIGS. 5-16 are merely examples, and that variations thereof or other safety checks are within the scope of the present technology. Also, while the steps of the methods are described below in a particular order, one or more of the steps can be performed in a different order or omitted, and the methods can include additional and/or alternative steps. Additionally, although the methods may be described below with reference to the embodiments of the present technology described herein, the methods can be performed with other embodiments of the present technology.

### i. Data Integrity/software Checks

FIG. 5 is a flowchart illustrating a method 500 for performing a handle apparatus data integrity check in accordance with various embodiments of the present technology. Data (e.g., binary files, programmed data) can be transmitted and stored to memory of a handle apparatus (e.g., the memory 134 of the handle apparatus 130) from, e.g., a control unit (the control unit 140 of FIG. 1, the control unit 240 of FIG. 2). The data can include identifying information (e.g., a serial number) and/or other information associated with the handle apparatus, the control unit, etc. In these and other embodiments, the data can include operating instructions, operating parameters, etc. for the handle apparatus, for an associated control unit, or a combination thereof. The method 500 can be executed to determine whether data stored to the memory of the handle apparatus is faulty (e.g., includes errors, is wrong) or is outdated. In some embodiments, the method 500 is performed by a processor of the handle apparatus (e.g., the processors 132 of the handle apparatus 130 of FIG. 1). The method 500 begins at block 502 by receiving an indication of a connection to power (e.g., to the power source 150 of FIG. 1, to the power source 250 of FIG. 2, to an internal power source such as a battery) and/or a control unit. For example, the indication can be a supply voltage provided from a power source and detected during a power-on routine of the handle apparatus.

At block 504, the method 500 continues by calculating (e.g., obtaining, computing, generating) a first checksum value based on the data stored to memory of the handle apparatus. The first checksum value can be calculated using various methods including, but not limited to, simple summations, cyclic redundancy checks (CRC), and/or hash functions (e.g., MD5, SHA-1, SHA-256). In some embodiments, calculating the first checksum value includes (a) scanning and/or reading out the data stored to the memory of the handle apparatus and (b) computing the first checksum value based at least in part on the scanned and/or read data.

At block 506, the method 500 continues by retrieving a second checksum value stored to the memory of the handle apparatus. The second checksum value can be associated with, —and/or stored independently of—the data upon which the first checksum value is calculated at block 504. In some embodiments, the second checksum value can be communicated and stored to memory of the handle apparatus with the data. As a specific example, the second checksum can be (i) calculated, external the handle apparatus, based on the data and (ii) thereafter communicated to the handle apparatus for storage on the handle apparatus along with the data.

At block 508, the method 500 continues by checking whether the first checksum value matches the second checksum value. In some embodiments, checking whether the first checksum value matches the second checksum value includes comparing the first checksum value to the second checksum value. The second checksum value can be an expected value for the first checksum value. Thus, when the first checksum values matches the second checksum value, the matching can indicate that the data stored in memory of the handle apparatus is devoid of errors (or at least is inclusive of errors that are correctable via, for example, error correction code and/or is not inclusive of uncorrectable errors). Thus, in the event that the first checksum value is equal to the second checksum value (block 508: Values are equal), the method 500 continues to block 510 by (a) determining that the integrity of the data stored to the memory of the handle apparatus is sound, and (b) enabling the handle apparatus to proceed (e.g., to a next safety check and/or to an enabled state for use in a medical procedure on a patient). In some embodiments, such as before enabling the handle apparatus to proceed to a next safety check and/or an enabled state for use in a medical procedure, the method 500 can further include (e.g., at block 510) communicating the first and/or the checksum value(s) to the control unit to, for example, verify the version of the software/firmware on the handle apparatus. Continuing with this example, the handle apparatus can thereby determine (e.g., based on a comparison between (i) the first and/or the second checksum value(s) and (ii) a third checksum value, such as a comparison performed at the control unit) whether its software/firmware is the most up-to-date version, whether its software/firmware is compatible with the particular control unit (e.g., with a current version of software/firmware used by the particular control unit), and/or the like.

On the other hand, when the first checksum value does not match the second checksum value, the disparity between the first checksum value and the second checksum value can indicate that errors are likely present in the data stored to the memory of the handle apparatus, or at least that the data stored to the memory does not fully align with/correspond to the data upon which the second checksum is based and/or computed. Thus, in the event that the first checksum value is not equal to the second checksum value (block 508: Value are not equal), the method 500 continues to block 512 by (a) determining that there are one or more issues associated with the data stored to the memory (e.g., that this data is corrupt), and (b) communicating the error (e.g., to a control unit, to a user/operator, to another computing system and/or display).

In some embodiments, the determination (e.g., whether the integrity of data stored to memory of the handle apparatus is sound or is corrupt) made at block 508 of the method 500 can be communicated to a physician (or other user/operator) via LED lights on the handle apparatus and/or the control unit, a screen on the handle apparatus and/or the control unit, an external display, and/or the like. Also, in the event that the first checksum value is not equal to the second checksum value (block 508: Values are not equal), the method 500 can include preventing or otherwise limiting powered and/or manual use of the handle apparatus in a medical procedure.

In some embodiments, the method 500 can include attempting to resolve errors identified with the data stored to the memory of the handle apparatus. For example, when the method 500 proceeds from block 508 to block 512, the method 500 can include (a) attempting to correct errors in the data, the first checksum value, and/or the second checksum value, and/or (b) repeating the method 500. Additionally, or alternatively, when the method 500 proceeds from block 508 to block 512, the method 500 can include (i) querying the control unit or another computing device for corrected data and/or a corrected checksum value, (ii) receiving the corrected data and/or the corrected checksum value, (iii) storing the corrected data and/or the corrected checksum value to memory (e.g., by overwriting the data and/or checksum that was determined to be compromised at block 508, and/or by saving the corrected data and/or the corrected checksum value to another location in memory), and/or (iv) repeating the method 500. When repeating the method 500, the method 500 can proceed from block 508 to block 510 in the event the first checksum value is equal to the second checksum value.

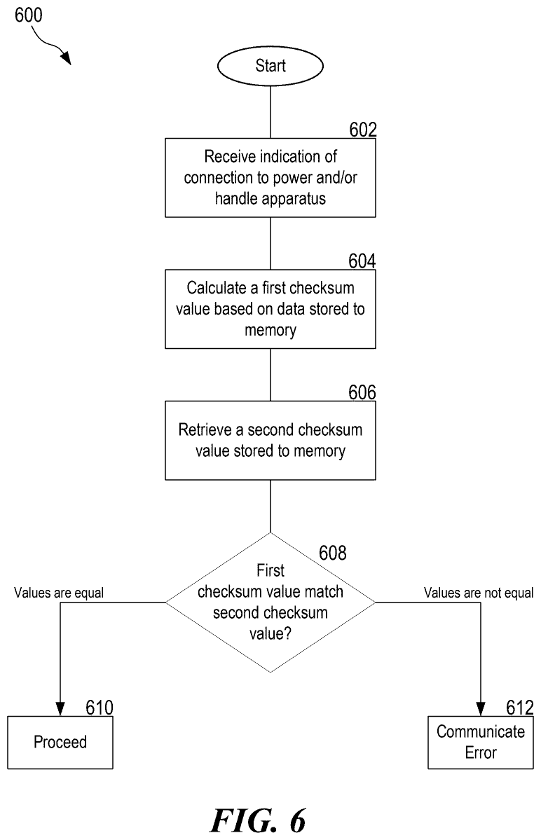

FIG. 6 is a flowchart illustrating a method 600 for performing a control unit data integrity check in accordance with various embodiments of the present technology. Data (e.g., binary files, programmed data) can be transmitted and stored to memory of a control unit (e.g., the memory 144 of the control unit 140 of FIG. 1) from, e.g., a handle apparatus (the handle apparatus 130 of FIG. 1, the surgical handle assembly 230 of FIG. 2) and/or another computing device (e.g., a remote server, such as over a wired or wireless communication network). The data can include identifying information (e.g., a serial number) and/or other information associated with the control unit, the handle apparatus, etc. In these and other embodiments, the data can include operating instructions, operating parameters, etc. for the control unit, for an associated handle apparatus, or a combination thereof. The method 600 can be executed to determine whether data stored to the memory of the control unit is faulty (e.g., includes errors, is wrong) or is outdated. In some embodiments, the method 600 is performed by a processor of the control unit (e.g., the processors 142 of the control unit 140 of FIG. 1). The method 600 begins at block 602 by receiving an indication of a connection to power (e.g., to the power source 150 of FIG. 1, to the power source 250 of FIG. 2) and/or a handle apparatus. For example, the indication can be a supply voltage provided from a power source and detected during a power-on routine of the control unit.

At block 604, the method 600 continues by calculating (e.g., obtaining, computing, generating) a first checksum value based on the data stored to memory of the control unit. The first checksum value can be calculated using various methods including, but not limited to, simple summations, cyclic redundancy checks (CRC), and/or hash functions (e.g., MD5, SHA-1, SHA-256). In some embodiments, calculating the first checksum value includes (a) scanning and/or reading out the data stored to the memory of the control unit, and (b) computing the first checksum value based at least in part on the scanned and/or read data.

At block 606, the method 600 continues by retrieving a second checksum value stored to the memory of the control unit. The second checksum value can be associated with—and/or stored independently of—the data upon which the first checksum value is calculated at block 604. In some embodiments, the second checksum value can be communicated and stored to memory of the control unit with the data. As a specific example, the second checksum can be (i) calculated, external the control unit, based on the data and (ii) thereafter communicated to the control unit for storage on the control unit along with the data.

At block 608, the method 600 continues by checking whether the first checksum value matches the second checksum value. In some embodiments, checking whether the first checksum value matches the second checksum value includes comparing the first checksum value to the second checksum value. The second checksum value can be an expected value for the first checksum value. Thus, when the first checksum values matches the second checksum value, the matching can indicate that the data stored in memory of the control unit is devoid of errors (or at least is inclusive of errors that are correctable via, for example, error correction code and/or is not inclusive of uncorrectable errors). Thus, in the event that the first checksum value is equal to the second checksum value (block 608: Values are equal), the method 600 continues to block 610 by (a) determining that the integrity of the data stored to the memory of the control unit is sound, and (b) enabling the control unit to proceed (e.g., to a next safety check and/or to an enabled state for use in a medical procedure). In some embodiments, such as before enabling the control unit to proceed to a next safety check and/or an enabled state for use in a medical procedure, the method 600 can further include (e.g., at block 510) communicating the first and/or the second checksum value to the handle apparatus and/or another computing device to, for example, verify the version of the software/firmware on the control unit. Continuing with this example, the control unit can thereby determine (e.g., based on a comparison between (i) the first and/or the second checksum value(s) and (ii) a third checksum value, such as a comparison performed at the handle apparatus or another computer device) whether its software/firmware is the most up-to-date version, whether its software/firmware is compatible with the particular handle apparatus (e.g., with a current version of software/firmware used by the particular handle apparatus), and/or the like.

On the other hand, when the first checksum value does not match the second checksum value, the disparity between the first checksum value and the second checksum value can indicate that errors are likely present in the data stored to the memory of the control unit, or at least that the data stored to the memory does not fully align with/correspond to the data upon which the second checksum is based and/or computed. Thus, in the event that the first checksum value is not equal to the second checksum value (block 608: Values are not equal), the method 600 continues to block 612 by (a) determining that there are one or more issues associated with the data stored to the memory (e.g., that this data is corrupt), and (b) communicating the error (e.g., to a handle apparatus, to another computing device and/or display, to a user/operator).

In some embodiments, the determination (e.g., whether the integrity of data stored to memory of the control unit is sound or corrupt) made at block 608 of the method 600 can be communicated to a physician (or other user/operator) via LED lights on the handle apparatus and/or the control unit, a screen on the handle apparatus and/or the control unit, an external display, and/or the like. Also, in the event that the first checksum value is not equal to the second checksum value (block 608: Values are not equal), the method 600 can include preventing or otherwise limiting powered and/or manual use of the handle apparatus (or another medical device coupled to the control unit) in a medical procedure.

In some embodiments, the method 600 can include attempting to resolve errors identified with the data stored to the memory of the control unit. For example, when the method 600 proceeds from block 608 to block 612, the method 600 can include (a) attempting to correct errors in the data, the first checksum value, and/or the second checksum value, and/or (b) repeating the method 600. Additionally, or alternatively, when the method 600 proceeds from block 608 to block 612, the method 600 can include (i) querying another computing device for corrected data and/or a corrected checksum value, (ii) receiving the corrected data and/or the corrected checksum value, (iii) storing the corrected data and/or the corrected checksum value to memory (e.g., by overwriting the data and/or checksum that was determined to be compromised at block 608, and/or by saving the corrected data and/or the corrected checksum value to another location in memory), and/or (iv) repeating the method 600. When repeating the method 600, the method 600 can proceed from block 608 to block 610 in the event the first checksum value is equal to the second checksum value.

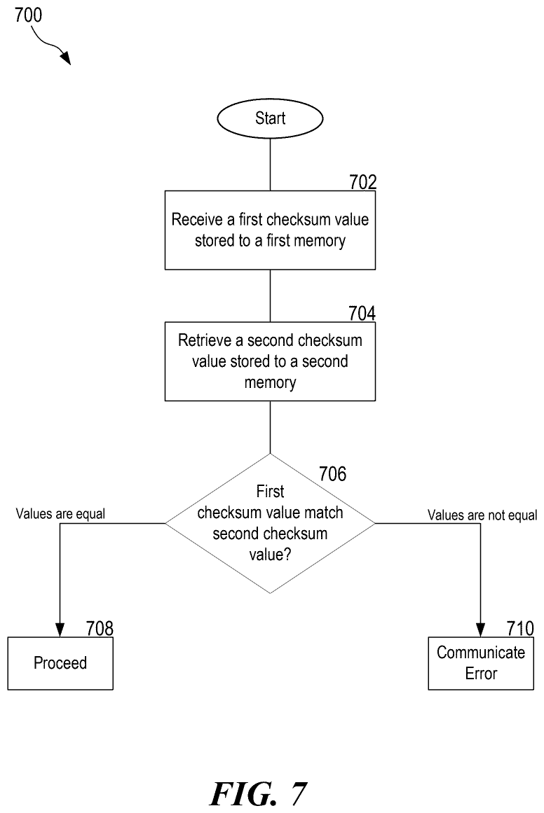

FIG. 7 is a flowchart illustrating a method 700 for performing a software/firmware check between a handle apparatus and a control unit in accordance with various embodiments of the present technology. The method 700 can be executed to confirm that, for example, the handle apparatus has the most up-to-date software and/or firmware, the control unit has the most up-to-date software and/or firmware, and/or the software/firmware used by the handle apparatus is compatible with the software/firmware used by the control unit. In some embodiments, the method 700 can be performed based at least in part on (a) power up of the handle apparatus, (b) power up of the control unit, and/or (c) a connection/communication (e.g., wired or wireless) being established between the handle apparatus and the control unit (e.g., the handle apparatus being plugged into and/or wirelessly connected with the control unit). The method 700 begins at block 702 by receiving (e.g., obtaining) a first checksum value stored to a first memory of one of a handle apparatus or a control unit. In some embodiments, the first checksum value is based at least in part on software, firmware, and/or other data stored in memory on the handle apparatus. In other embodiments, the first checksum value is based at least in part on software, firmware, and/or other data stored in memory on the control unit.

At block 704, the method 700 continues by retrieving a second checksum value stored to a second memory of the other of the handle apparatus or the control unit. In some embodiments, the second checksum value corresponds to (a) a most up-to-date version of software, firmware, and/or other data available for the one of the handle apparatus or the control unit, or (b) a version of software, firmware, and/or other data for the one of the handle apparatus or the control unit that is compatible with a current version of software, firmware, and/or other data currently used by the other of the handle apparatus or the control unit.

At block 706, the method 700 continues by checking whether the first checksum value matches the second checksum value. In some embodiments, checking whether the first checksum value matches the second checksum value includes comparing the first checksum value to the second checksum value. The second checksum value can be an expected value for the first checksum value. Thus, when the first checksum value matches the second checksum value, the matching can indicate, for example, that (i) a current version of software/firmware employed by the one of the handle apparatus of the control unit is a most up-to-date version and/or (ii) the current version of software/firmware is compatible with a current version of software/firmware employed by the other of the handle apparatus or the control unit. Thus, in the event that the first checksum value is equal to the second checksum value (block 706: Values are equal), the method 700 continues to block 708 by (a) determining that the software/firmware corresponding to the first checksum value is an up-to-date version and/or compatible and (b) enabling the handle apparatus and/or the control unit to proceed (e.g., to a next safety check and/or to an enabled state for use in a medical procedure).

On the other hand, when the first checksum value does not match the second checksum value, the disparity between the first checksum value and the second checksum value can indicate, for example, that (i) a current version of software/firmware employed by the one of the handle apparatus of the control unit is not a most up-to-date version and/or (ii) the current version of software/firmware is not compatible with a current version of software/firmware employed by the other of the handle apparatus or the control unit. Thus, in the event that the first checksum value is not equal to the second checksum value (block 706: Values are not equal), the method 700 continues to block 708 by (a) determining that the software/firmware corresponding to the first checksum value is not an up-to-date version and/or compatible and (b) communicating the error (e.g., to the one of the handle apparatus or the control unit, to a user/operator, to another computing system and/or display).

In some embodiments, the determination (e.g., whether the software/firmware employed by the one of the handle apparatus or the control unit is a most up-to-date version and/or compatible with software/firmware employed by the other of the handle apparatus or the control unit) can be communicated to a physician (or other user/operator) via LED lights on the handle apparatus and/or the control unit, a screen on the handle apparatus and/or the control unit, an external display, and/or the like. In some embodiments, in the event that the first checksum value is not equal to the second checksum value (block 706: Values are not equal), the method 700 can include preventing or otherwise limiting powered and/or manual use of the handle apparatus in a medical procedure.

In some embodiments, software versioning and/or incompatibility can be resolved by, for example, updating the data/software/firmware stored to the memory of the one of the handle apparatus or the control unit. For example, in the event that current software/firmware on a handle apparatus is not a most up-to-date version and/or is incompatible with current software/firmware employed by the control unit, the handle apparatus and/or the control unit can implement a software/firmware update to bring the software/firmware/other data employed by the handle apparatus and/or the control unit to a most up-to-date version and/or to compatibility with one another. Thereafter, the method 700 can be repeated, and the method 700 can proceed from block 706 to block 708 in the event the first checksum value is equal to the second checksum value.

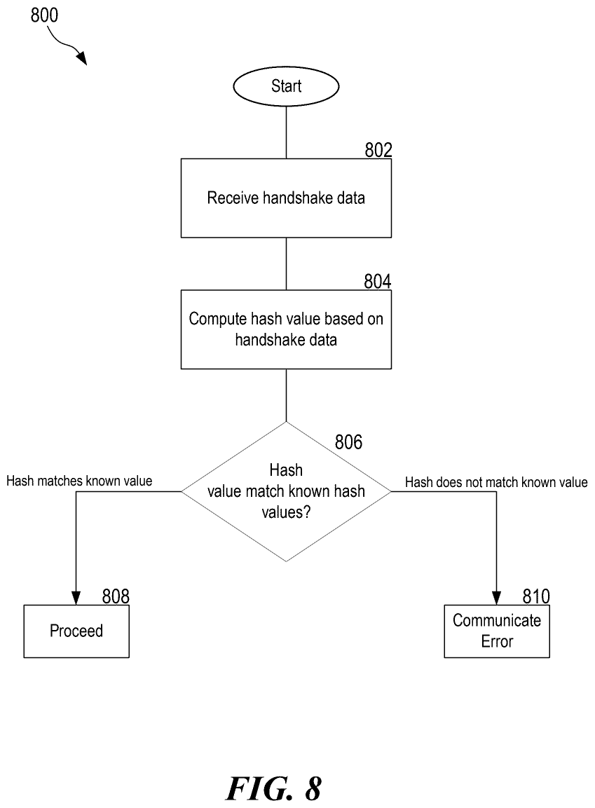

FIG. 8 is a flowchart illustrating a method 800 for performing a handshake protocol between a handle apparatus and a control unit in accordance with various embodiments of the present technology. The method 800 can be executed to confirm that, for example, the handle apparatus and the control unit are in sync and/or can properly communicate with one another. In some embodiments, the method 800 can be performed based at least in part on (a) power up of the handle apparatus, (b) power up of the control unit, and/or (c) a connection/communication (e.g., wired or wireless) being established between the handle apparatus and the control unit (e.g., the handle apparatus being plugged into and/or wirelessly connected with the control unit). The method 800 begins at block 802 by receiving handshake data. In some embodiments, the handshake data is received at the control unit and from the handle apparatus. In other embodiments, the handshake data is received at the handle apparatus and from the control unit. The handshake data can include identifying information (e.g., a serial number), a private key, and/or the like related to the handle apparatus and/or the control unit.

At block 804, the method 800 continues by computing a hash value based at least in part on the handshake data. In some embodiments, the hash value can be computed using cryptographic hash functions, hash-based message authentication code (HMAC), digital signatures, key derivation functions (KDFs), custom hashing algorithms, and/or the like.

At block 806, the method 800 continues by checking whether the computed hash value matches a known hash value. In some embodiments, checking whether the computed hash value matches the known hash value includes comparing the hash value computed at block 804 to the known hash value. As a specific example, the known hash value can be one of several known hash values in a lookup table (e.g., stored to the handle apparatus and/or the control unit), and checking whether the computed hash value matches the known hash value can include comparing the known hash value to entries of hash values in the lookup table to determine whether the computed hash value matches one of the entries in the table. In some embodiments, the known hash value(s) can correspond to compatible handle apparatus(es) and/or compatible control unit(s). As such, when the computed hash value matches a known hash value, this can indicate compatibility between the handle apparatus and the control unit, or can otherwise indicate that the handle apparatus and the control unit are in sync and/or can properly communicate with one another. Thus, in the event that the computed hash value matches the known hash value (block 806: Hash matches known value), the method 800 continues to block 808 by (a) determining that the handle apparatus and the control unit are synchronized and/or able to properly communicate with one another, and (b) enabling the handle apparatus and the control unit to proceed (e.g., to a next safety check and/or to an enabled state for in a medical procedure).

On the other hand, when the computed hash value does not match a known hash value, this can indicate incompatibility between the handle apparatus and the control unit, or can otherwise indicate that the handle apparatus and the control unit are not in sync and/or are not able to properly communicate with one another. Thus, in the event that the computed hash value does not match the known hash value (block 806: Hash does not match known value), the method 800 continues to block 810 by (a) determining that the handle apparatus and the control unit are not synchronized and/or not able to properly communicate with one another, and (b) communicating the error (e.g., to the handle apparatus when the control unit performs the method 800, to the control unit when the handle apparatus performs the method 800, to a user/operator, to another computing system and/or display). In some embodiments, the determination (e.g., whether the handle apparatus and the control unit are in sync and/or can properly communicate) made at block 806 of the method 800 can be communicated to a physician (or other user/operator) via LED lights on the handle apparatus and/or the control unit, a screen on the handle apparatus and/or the control unit, an external display, and/or the like. Also, in the event that the computed hash value does not match the known hash value (block 806: Hash does not match known value), the method 800 can include preventing or otherwise limiting powered and/or manual use of the handle apparatus and/or the control unit in a medical procedure.

It is appreciated that blocks 802-810 merely represent one example method of performing a handshake protocol between a handle apparatus and a control unit. In other words, computing and comparing hash values is not required to properly establish communication between the handle apparatus and the control unit. For example, in some embodiments, a method of performing a handshake protocol between a handle apparatus and a control unit, performed by one of the handle apparatus or the control unit, can include (i) receiving handshake data from another of the handle apparatus or the control unit, (ii) transmitting, in response to the received handshake data, an acknowledgement signal to the another of the handle apparatus or the control unit, (iii) exchanging communication parameters with the other of the handle apparatus or the control unit, and (iv) establishing communication with the other of the handle apparatus or the control unit based at least in part on the exchanged communication parameters. As non-limiting examples, the communication parameters can include a data transfer rate, an encryption scheme, an error-checking protocol, and/or the like.

### ii. Voltage Checks

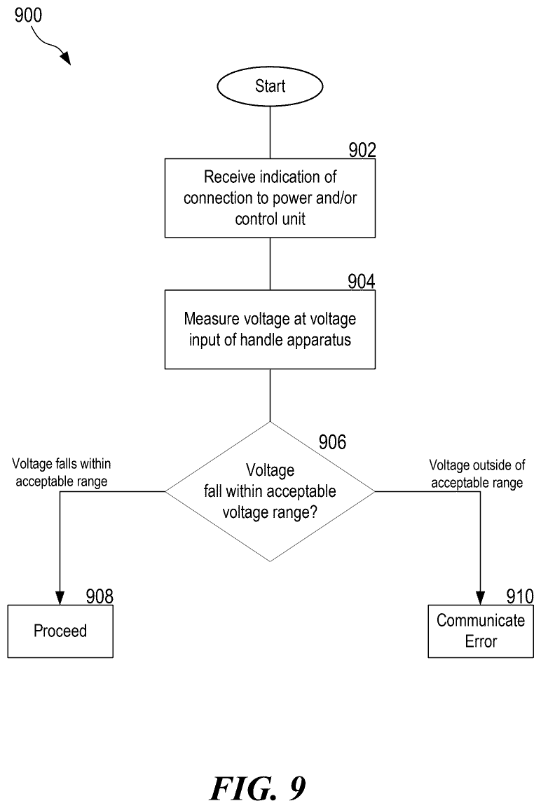

FIG. 9 is a flowchart illustrating a method 900 for performing a handle apparatus voltage input check in accordance with various embodiments of the present technology. In some embodiments, the method 900 is executed by processors/microcontrollers of a handle apparatus (e.g., the processors 132 of the handle apparatus 130 of FIG. 1) to confirm that a voltage supplied to the handle apparatus is within an expected, acceptable, and/or safe range and/or whether the handle apparatus is viable for use in a medical procedure. The method 900 begins at block 902 by receiving an indication of connection to power (e.g., to the power source 150 of FIG. 1, to the power source 250 of FIG. 2, to an internal power source such as a battery) and/or a control unit. For example, the indication can be a supply voltage provided from a power source and detected during a power-on routine of the handle apparatus.

At block 904, the method 900 continues by measuring voltage at a voltage input of the handle apparatus. In some embodiments, the handle apparatus includes a resistor with a known resistance value that is placed in series with a voltage supply line of the handle apparatus. Continuing with this example, the handle apparatus can (e.g., using an analog-to-digital converter) measure a voltage drop across the resistor and supply the voltage drop measurement to a microcontroller/processor of the handle apparatus. Given the known resistance value of the resistor and the measured voltage drop across the resistor, the microcontroller/processor of the handle apparatus can determine the voltage at the voltage input of the handle apparatus.

At block 906, the method 900 continues by determining whether the measured voltage at the voltage input falls within an acceptable voltage range. In some embodiments, determining whether the measured voltage at the voltage input falls within the acceptable voltage range includes comparing the measured value to one or more voltage values of a range of acceptable voltage values and determining whether the measured voltage value falls within or outside of the range. In the event that the measured voltage falls within the acceptable voltage range (block 906: Voltage falls within acceptable range), the method 900 continues to block 908 by (a) determining that the voltage supplied to the handle apparatus is safe/acceptable and/or that the handle apparatus is viable for use, and (b) allowing the handle apparatus to proceed (e.g., to a next safety check and/or to an enabled state for use in a medical procedure).

On the other hand, in the event that the measured voltage is outside of the acceptable voltage range (block 906: Voltage outside of acceptable range), the method 900 continues to block 910 by (a) determining that the voltage supplied to the handle apparatus is not safe/acceptable and/or that the handle apparatus is not viable for use, and (b) communicating the error (e.g., to the control unit, to a user/operator, to another computing system and/or display). In some embodiments, the determination (e.g., whether the voltage supplied to the handle apparatus is within a safe, acceptable, and/or expected range) made at block 906 of the method 900 can be communicated to a physician (or another user/operator) via LED lights on the handle apparatus and/or the control unit, a screen on the handle apparatus and/or the control unit, an external display, and/or the like. Also, in the event that the measured voltage value at the input is outside of the safe, acceptable, and/or acceptable range (block 906: Voltage outside of acceptable range), the method 900 can include preventing or otherwise limiting powered and/or manual use of the handle apparatus in a medical procedure.

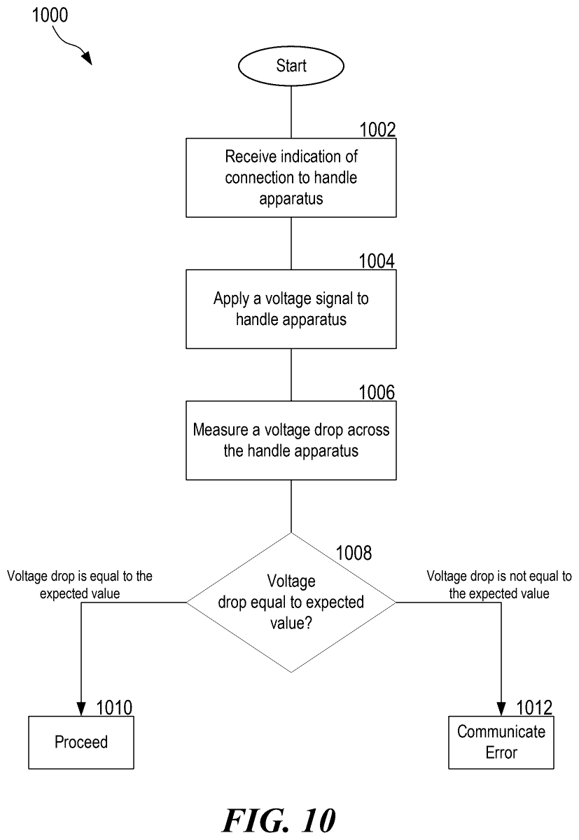

FIG. 10 is a flowchart illustrating a method 1000 for performing a voltage input check or power negotiation in accordance with various embodiments of the present technology. In some embodiments, processors of a control unit (e.g., the processors 142 of the control unit 140 of FIG. 1) can execute the method 1000 to confirm which handle apparatus is connected to the control unit (e.g., to confirm an identity and/or operation parameters of the handle apparatus). It is appreciated that the method 1000 (or other power negotiation methods) can be performed or implemented as a part of a handshake protocol (e.g., the handshake protocol of method 800). The method 1000 begins at block 1002 by receiving an indication of connection (e.g., a wired or wireless connection) to a handle apparatus.

At block 1004, the method 1000 continues by applying a voltage signal to the handle apparatus. In some embodiments, the voltage signal is applied across two or more pins facilitating the connection between the handle apparatus and the control unit. In these and other embodiments, the voltage signal is applied across one or more resistors, such as one or more resistors placed in series with the two or more pins. The voltage signal applied at block 1004 can be a lower voltage value than a voltage level required for operation of the handle apparatus (e.g., to reduce, minimize, or eliminate a risk of damaging electronics of the handle apparatus).

At block 1006, the method 1000 continues by measuring a voltage drop across the handle apparatus. For example, the voltage signal applied at block 1004 above can be applied to a first connection pin that facilitates a connection between the handle apparatus and the control unit, and the control unit can measure a voltage level of a returning signal on a second connection pin that is electrically coupled to the first connection pin and that also facilitates a connection between the handle apparatus and the control unit. Continuing with this example, the voltage level measured on the second connection pin can be compared against the voltage level of the voltage signal applied to the first connection pin at block 1004 above to determine a voltage drop across the two connection pins. As discussed above, the voltage drop can be attributable to one or more resistors placed in series with the two connection pins. Thus, the voltage drop measured at block 1006 can indicate resistance values of the resistors. In some embodiments, measuring the voltage drop across the handle apparatus can include determining or computing the resistance values corresponding to the resistors (e.g., a total resistance, such as a resistance inclusive of the resistance of the resistor(s), connection pin resistance, transmission line resistance, etc.).

At block 1008, the method 1000 continues by determining whether the measured voltage drop at block 1006 is equal to an expected value. Determining whether the measured voltage drop is equal to an expected value can include comparing the measured voltage drop to an expected value. The expected value can be a value unique to a particular handle apparatus or a particular type of handle apparatuses. Additionally, or alternatively, the expected value can be one of a plurality of expected values corresponding to a plurality of known/compatible handle apparatuses and/or a plurality of known/compatible types of handle apparatuses. For example, different handle apparatuses/handle apparatus types can be associated with different expected voltage drop values. As a specific example, different handle apparatuses/handle apparatus types can include different sets of one or more resistors (with differing total resistance values) in series with their connection pins. Continuing with this example, each of the total resistance values of the different sets of one or more resistors can be associated with a different voltage drops across the connection pins of the respective handle apparatus when a same voltage signal is applied to the connection pins. In at least some of these embodiments, the control unit can store a lookup table that associates (a) each handle apparatus/handle apparatus type in a list of possible/compatible handle apparatuses/handle apparatus types with (b) its expected voltage drop value (or with a corresponding range of expected voltage drop values). Continuing with this example, determining whether the measured voltage drop at block 1006 is equal to an expected value can include determining whether the measured voltage drop matches a voltage drop value (or a range of voltage drop values) stored in the lookup table. In other embodiments, the control unit can store a single expected value or a single range of expected values, and determining whether the measured voltage drop at block 1006 is equal to an expected value can include determining whether the measured voltage drop is equal to the single expected value or falls within a range of expected values. the correct or expected handle apparatus is connected to the control unit. In still other embodiments, the expected value or a plurality of expected values stored by the control unit can be associated with specific operating and/or power parameters. Thus, a comparison (e.g., using a lookup table) of the measured voltage drop value from block 1006 with the expected voltage drop value(s) at block 1008 can identify appropriate operating and/or power parameters to supply to the connected handle apparatus. In some embodiments, appropriate operating and/or power parameters for a handle apparatus that can be identified using the measured voltage drop value from block 1006 can include a higher, lower, or equivalent voltage level than or as the voltage level of the voltage signal applied at block 1004.

When the measured voltage drop equals the expected voltage drop or falls within a range of expected voltage drop values, this can (a) indicate that the handle apparatus is known/compatible with the control unit and/or (b) indicate appropriate operating/power parameters (e.g., input voltage) to supply the handle apparatus. Thus, in the event that the measured voltage drop is equal to the expected value (block 1008: Voltage drop is equal to the expected value), the method 1000 continues to block 1010 by (a) determining that the handle apparatus is known and/or compatible with the control unit; (b) identifying (and ultimately supply) appropriate operating/power parameters to the handle apparatus; and/or (c) allowing the control unit and the handle apparatus to proceed (e.g., to a next safety check and/or to an enabled state for use in a medical procedure).

On the other hand, in the event that the measured voltage drop is not equal to the expected value(s) (block 1008: Voltage drop is not equal to the expected value), the method 1000 continues by (a) determining that the handle apparatus is not known and/or is not compatible with the control unit; (b) determining that the appropriate operating/power parameters for the handle apparatus could not be identified; and/or (c) communicating the error (e.g., to the handle apparatus, to a user/operator, to another computing system and/or display). In some embodiments, the determination (e.g., whether the handle apparatus is known and/or compatible, whether appropriate operating/power parameters could be identified) can be communicated to a physician (or another user/operator) via LED lights on the handle apparatus and/or the control unit, a screen on the handle apparatus and/or the control unit, an external display, and/or the like. Also, in the event that the measured voltage drop does not match or correspond to an expected value or range of expected values (block 1008: Voltage drop is not equal to the expected value), the control unit can prevent or otherwise limit powered and/or manual actuation of the handle apparatus in a medical procedure (e.g., by preventing power delivery to the handle apparatus, by limiting power delivery to the handle apparatus to levels insufficient to conduct a medical procedure).

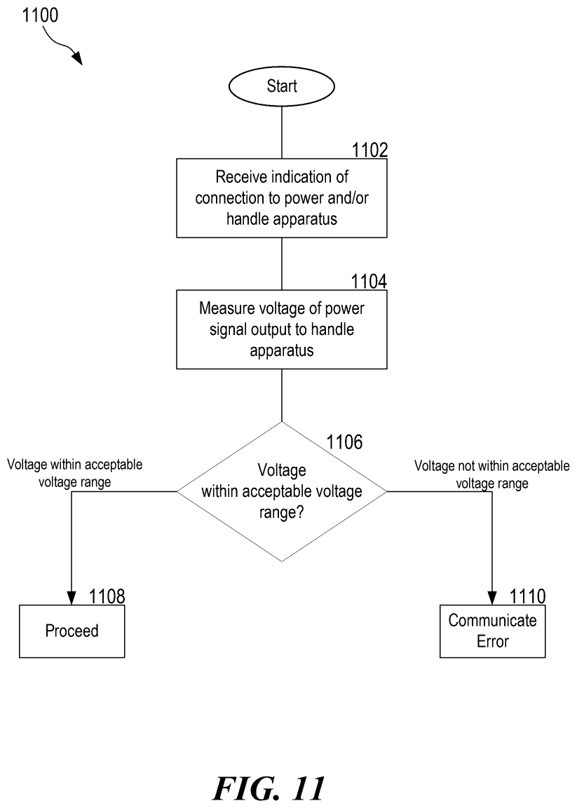

FIG. 11 is a flowchart illustrating a method 1100 for performing a control unit output power check in accordance with various embodiments of the present technology. In some embodiments, processors of a control unit (e.g., the processors 142 of the control unit 140 of FIG. 1) can execute the method 1100 to confirm that the control unit is able to output/supply (or is currently outputting/supplying) appropriate power levels (e.g., to a connected handle apparatus). In some embodiments, the method 1100 begins at block 1102 by receiving an indication of connection to power (e.g., to the power source 150 of FIG. 1, to the power source 250 of FIG. 2, to an internal power source such as a battery, and/or to a handle apparatus or another device.

At block 1104, the method 1100 can continue (or, in some embodiments, begin) by measuring a voltage level of a power signal output to a connected handle apparatus or another connected device. In some embodiments, the voltage level can be measured based on a voltage drop across a resistor (e.g., a resistor of the control unit having a known resistance, a resistor of a connected handle apparatus or of another connected device having a known resistance). For example, a resistor having a known resistance value can be placed in series with a power output line of the control unit, and the control unit can (e.g., using an analog-to-digital converter) measure a voltage drop across the resistor and supply the voltage drop measurement to a microcontroller/processor of the control unit. Given the known resistance value of the resistor and the measured voltage drop across the resistor, the microcontroller/processor of the handle apparatus can determine the voltage at the voltage input of the handle apparatus. In some embodiments, the method 1000 can include measuring other power parameters (e.g., amperage, wattage) of the power signal in addition to or in lieu of measuring the voltage level.

At block 1106, the method 1100 continues by determining whether the measured voltage level (and/or other measured power parameters) is within an acceptable voltage range. In some embodiments, determining whether the measured voltage level is within the acceptable voltage range includes comparing the measured voltage value to one or more voltage values of a range of acceptable voltage values and determining whether the measured voltage value falls within or outside of the range. In the event that the measured voltage is within the acceptable voltage range (block 1106: Voltage within acceptable voltage range), the method 1100 continues to block 1108 by (a) determining that the power signal/voltage output by the control unit and/or supplied to a connected handle apparatus or other connected device is safe/acceptable and/or that the control unit is viable for use, and (b) allowing the control unit to proceed (e.g., to a next safety check, to continue supply power to the connected handle apparatus/device, and/or to an enabled state for use in a medical procedure).

On the other hand, in the event that the measured voltage (and/or other measured power parameters) is not within the acceptable voltage range (block 1106: Voltage not within acceptable voltage range), the method 1100 continues to block 1110 by (a) determining that the power signal/voltage output by the control unit and/or supplied to a connected handle apparatus or other connected device is not safe/acceptable and/or that the control unit is not viable for us, and (b) communicating the error (e.g., to the handle apparatus, to a user/operator, to another computing system and/or display). In some embodiments, the determination (e.g., whether the voltage and/or other power parameters of a power signal output by the control unit is safe/acceptable, whether the control unit is viable for use) can be communicated to a physician (or another user/operator) via LED lights on the handle apparatus and/or the control unit, a screen on the handle apparatus and/or the control unit, an external display, and/or the like. Also, in the event that the measured voltage or other measured power parameter is not within an acceptable range (block 1106: Voltage not within acceptable voltage range), the control unit can prevent or otherwise limit (a) power output from the control unit, (b) powered and/or manual actuation of a connected handle apparatus or other device, and/or (c) use of the control unit (e.g., in a medical procedure).

iii. Motor Encoder Functionality Checks

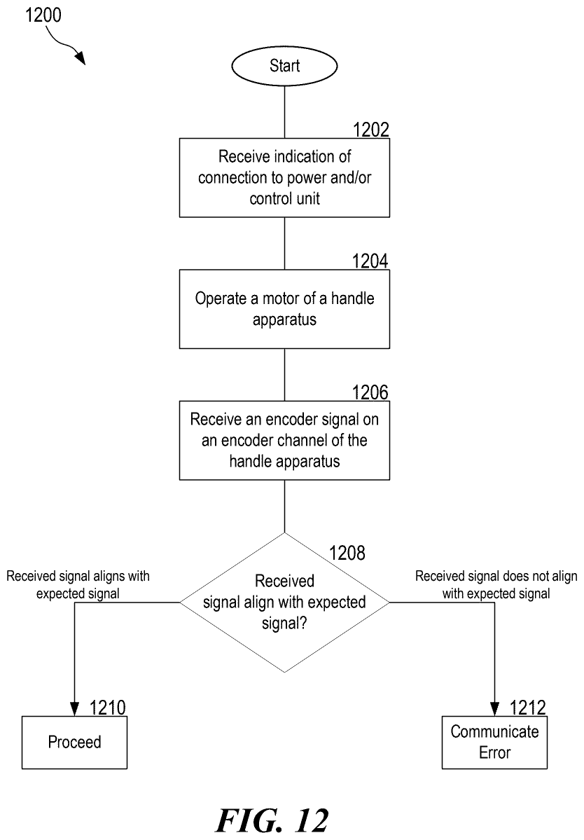

FIG. 12 is a flowchart illustrating a method 1200 for performing a handle apparatus motor encoder functionality check in accordance with various embodiments of the present technology. In some embodiments, processors/microcontrollers of a handle apparatus (e.g., the processors 132 of the handle apparatus 130 of FIG. 1) or processors of a control unit (e.g., the processors 142 of the control unit 140 of FIG. 1) can execute the method 1200 to confirm whether one or more encoders included in the handle apparatus are functioning properly. The method 1200 begins at block 1202 by receiving (i) an indication of connection to power (e.g., to the power source 150 of FIG. 1, to the power source 250 of FIG. 2, to an internal power source such as a battery), such as to initiate a power-up/initialization routine of the handle apparatus, and/or (ii) an indication of connection (e.g., a wired or wireless connection) between the handle apparatus and a control unit.

At block 1204, the method 1200 continues by operating a motor of the handle apparatus (e.g., the electric motor 319 of the surgical handle assembly 230). The motor can be operated during a homing/initialization period of the surgical device during which a drive assembly is positioned in a starting position or initialized state. In some embodiments, the motor is operated according to a specific pattern, such as rotating in one direction, then rotating in the opposite direction to return the motor to an original, known, or initialized state.

At block 1206, the method 1200 continues by receiving an encoder signal on the handle apparatus (e.g., over an encoder channel of the handle apparatus). As discussed above with reference to FIG. 3, the surgical handle assembly 230 can include one or more encoders (e.g., a linear encoder for measuring position, a rotary encoder for measuring rotation) operably coupled to the electrical motor 319, and encoder signals can be received over one or more encoder channels included in the surgical handle assembly 230. An encoder signal can indicate a position, orientation, etc. of a component of—or attached to—the motor. In some embodiments, the handle apparatus includes two, three, four, or more encoder channels.

At block 1208, the method 1200 continues by determining whether the received encoder signal aligns with an expected encoder signal. The determination can be based on communication between the motor driver and a microcontroller (e.g., the microcontroller 480 of FIG. 4A) onboard the handle apparatus. In these and other embodiments, the expected signal can correspond to a specific pattern of operation performed at block 1204 and/or the type of encoder from which the encoder signal is received. For example, at block 1204, the motor can be operated to rotate in one direction and then the other direction to return to the original state. Continuing with this example, the expected encoder signal can therefore include a rise and fall in the value of the position, orientation, etc. indicated by the encoder signal. In some embodiments, determining whether the received encoder signal aligns with the expected encoder signal can include comparing the received encoder signal to the expected encoder signal.

When the received encoder signal aligns with the expected encoder signal (e.g., within a margin of error), the alignment can indicate that the encoder is functioning properly. Thus, in the event that the received encoder signal aligns with the expected signal (block 1208: Received signal aligns with expected signal), the method 1200 continues to block 1210 by (a) determining that the corresponding encoder is functioning properly and/or (b) allowing the handle apparatus to proceed (e.g., to a next safety check and/or to an enabled state for use in a medical procedure). On the other hand, in the event that the received signal does not align with the expected signal (block 1208: Received signal does not align with expected signal), the method 1200 continues to block 1212 by (a) determining that the corresponding encoder is not functioning properly and (b) communicating the error (e.g., to the control unit, such as when the handle apparatus conducts the method 1200; to the handle apparatus, such as when the control unit conducts the method 1200, to a user/operator, to another computing system and/or display). In some embodiments, the determination (e.g., whether an encoder is functioning properly and/or whether a received encoder signal aligns with an expected encoder signal) can be communicated to a physician (or another user/operator) via LED lights on the handle apparatus and/or the control unit, a screen on the handle apparatus and/or the control unit, an external display, and/or the like. Also, in the event that the received encoder signal does not align with the expected encoder signal (block 1208: Received signal does not align with expected signal), the handle apparatus and/or the control unit can prevent or otherwise limit powered and/or manual actuation of the handle apparatus in a medical procedure.

### iv. Reload Assembly Checks

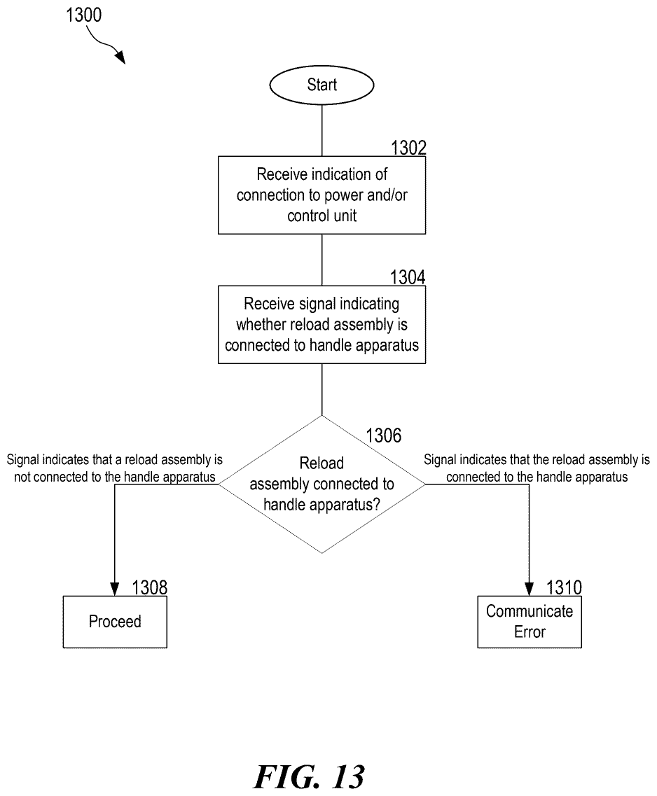

FIG. 13 is a flowchart illustrating a method 1300 for performing a reload assembly check in accordance with various embodiments of the present technology. In some embodiments, the processors of a handle apparatus (e.g., the processors 132 of the handle apparatus 130 of FIG. 1) or processors of a control unit (e.g., the processors 142 of the control unit 140 of FIG. 1) can execute the method 1300 to confirm that the handle apparatus is not connected a reload assembly (e.g., the reload assembly 120 of FIG. 1, the surgical reloadable cartridge assembly 220 of FIG. 2) when initially connected to power, initially connected to the control unit, and/or initially powered on. Ensuring that the handle apparatus is not initially connected to a reload assembly may be desirable, for example, to (a) help prevent reusing a reload assembly (e.g., a reloadable cartridge assembly that has previously been fully or partially fired and therefore presents a risk of cutting tissue without stapling) and/or (b) help ensure that a new/sterilized reload assembly is used. The method 1300 begins at block 1302 by receiving an indication of connection to power (e.g., to the power source 150 of FIG. 1, to the power source 250 of FIG. 2, to an internal power source such as a battery), an indication of powerup of the handle apparatus, and/or an indication of a connection (e.g., wired or wireless) to the control unit.

At block 1304, the method 1300 continues by receiving a signal indicating whether a reload assembly is connected to the handle apparatus. In some embodiments, the signal is received from a switch included in the handle apparatus (e.g., the switch 483 on the circuit board 355 of FIG. 4B) that can flip between two states depending on whether a reload assembly is connected to the handle apparatus.

At block 1306, the method 1300 continues by determining, based at least in part on the signal received at block 1304, whether a reload assembly is connected to the handle apparatus. In the event that the received signal indicates that a reload assembly is not connected to the handle assembly (block 1306: Signal indicated that a reload assembly is not connected to the handle apparatus), the method 1300 continues to block 1308 by (a) determining that a reload assembly is currently not connected to the handle apparatus, and (b) allowing the handle apparatus to proceed (e.g., to a next safety check and/or to an enabled state for use in a medical procedure). On the other hand, in the event that the received signal indicates that a reload assembly is connected to the handle apparatus (block 1306: Signal indicated that the reload assembly is connected to the handle apparatus), the method 1300 continues to block 1310 by (a) determining that a reload assembly is currently connected to the handle apparatus and (b) communicating the error (e.g., to the control unit, such as when the handle apparatus conducts the method 1300; to the handle apparatus, such as when the control unit conducts the method 1300; to a user/operator; to another computing system and/or display). In some embodiments, the determination (e.g., whether a reload assembly is currently connected to the handle apparatus) can be communicated to a physician (or other user/operator) via LED lights on the handle apparatus and/or the control unit, a screen on the handle apparatus and/or the control unit, an external display, and/or the like. Also, in the event that there is currently a reload assembly connected to the handle apparatus, the handle apparatus and/or the control unit can prevent or otherwise limit powered and/or manual actuation of the handle apparatus in a medical procedure. For example, electrical operation of the reload assembly can be restricted. As another example, distal advancement of a drive assembly in the handle apparatus and/or a blade assembly in the reload assembly can be prohibited, at least until after the handle apparatus is returned to an initialized or “homed” state. In some embodiments, as discussed in further detail below, the handle apparatus may allow the physician to use the selector lever 217 (e.g., to retract a drive assembly and/or a blade assembly proximally) when powered and/or manual actuation of the handle apparatus is limited.

As discussed above, a reload assembly may be connected to the handle apparatus upon connection to power, upon powerup, and/or upon connection (e.g., wired or wireless) to the control unit, which can result in the method 1300 proceeding from block 1306 to block 1310 for various reasons. There are several scenarios in which this can occur. For example, while operating the handle apparatus and an attached reload assembly in a medical procedure, a power loss event can occur mid-firing of the reload assembly (e.g., from a malfunction of the control unit, the handle apparatus, or an associated system/power supply; accidental disconnection from power, such as from a healthcare professional accidentally disconnecting a power cord from the outlet; a power outage; and/or the like), after which power and/or a connection to the control unit can be restored to the handle apparatus. As another example, to ensure that the reload assembly connected to the handle apparatus is new (e.g., sterile and/or unfired), the system may require that the handle apparatus be powered on prior to attaching a reload assembly to the handle apparatus. Thus, the method 1300 can proceed from block 1306 to block 1310 in the event that a physician may have (e.g., in haste) connected a new or used reload assembly to the handle apparatus before powering on the handle apparatus and/or connecting the handle apparatus to a control unit.

In either of the above scenarios, the error communicated at block 1310 can be resolved. For example, the physician can (i) disconnect the reload assembly from the handle apparatus, (ii) return the handle apparatus to an initialized or “homed” state, which can be done manually, electrically, and/or autonomously after powerup of the handle apparatus, (iii) power cycle the handle apparatus by disconnecting and reconnecting the handle apparatus from and to power (after removal of the reload assembly), and/or (iii) connect either the same reload assembly or a new reload assembly to the handle apparatus. In some embodiments, the handle apparatus includes safety components that can mechanically prevent or at least resist/hinder disconnecting a reload assembly from the handle apparatus when the reload assembly and the handle apparatus are in a mid-fired or fully fired state. For example, when the handle apparatus is in a mid-fired or fully fired state, mechanical components can require the reload assembly and/or the handle assembly to return to the “homed” state (e.g., via proximal motion/retraction) before the mechanical components enable removal of the reload assembly form the handle apparatus. Such safety components can prevent/hinder accidental detachment of the reload assembly from the handle apparatus while the reload assembly is inserted in a patient. To allow the physician to properly disconnect the reload assembly when the handle apparatus is in the mid-fired or fully fired state, the handle apparatus can allow the physician to configure the handle apparatus from the firing state to a non-firing state, such as by using the selector lever 217 (FIGS. 2 and 3) to electrically retract a gear rack or other transmission component of the handle apparatus (e.g., retract the gear rack proximally in a direction extending from the reload assembly and away from the patient). Subsequently (e.g., once the handle apparatus has been returned to the “homed” position), the reload assembly can be properly disconnected from the handle assembly, such as by pressing a reload detach button on the handle apparatus.

In some cases, however, it may be desirable to disconnect a reload assembly from the handle apparatus when the handle assembly and/or the reload assembly are in a mid-fired or fully fired state. In such cases, the physician may forcefully disconnect the reload assembly by, for example, using a distal force (e.g., brute force) to overpower the aforementioned safety components. When this occurs, the handle apparatus may detect the forceful removal of the reload assembly via a switch (e.g., the switch 483 of FIG. 4B) in the handle apparatus. Thus, at least in part in response to the forced removal of the reload assembly from the handle apparatus, the handle apparatus can lock itself out from further use, at least until the handle apparatus power cycled and/or is otherwise returned to the homed state. Subsequently, the disconnected reload assembly or a new reload assembly may be connected to the handle apparatus.

### v. Error Code Checks

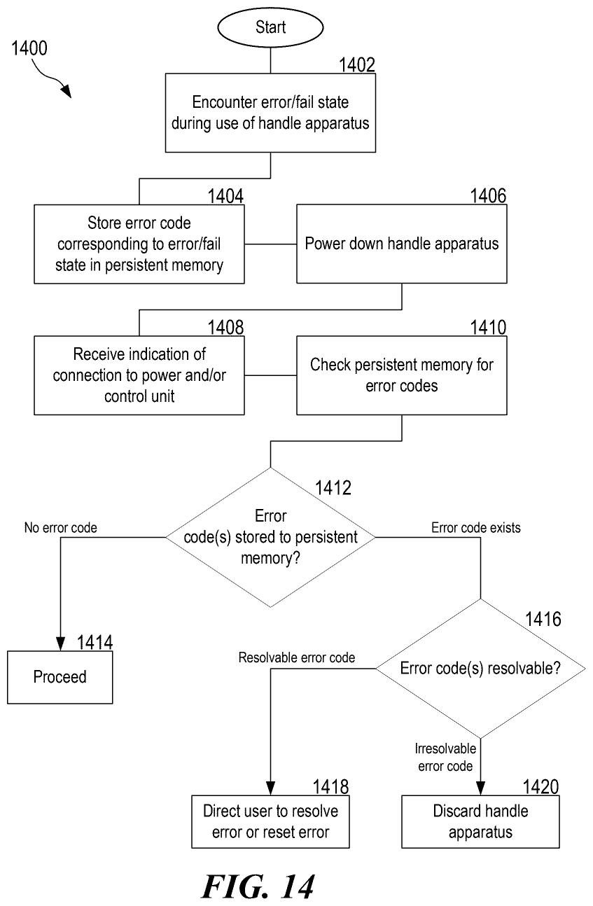

FIG. 14 is a flowchart illustrating a method 1400 for performing a handle apparatus error code check in accordance with various embodiments of the present technology. In some embodiments, processors of a handle apparatus (e.g., the processors 132 of the handle apparatus 130 of FIG. 1) can execute the method 1400 to check for and respond to any error codes retained by the handle apparatus from, for example, a previous power cycle.

The method 1400 begins at block 1402 by encountering an error or fail state during use and/or a power cycle of the handle apparatus. In some embodiments, the error or fail state can correspond to use of a manual retraction override of the handle apparatus, reaching a predetermined maximum number of uses/firings of the handle apparatus or a reload assembly, a maximum amount of time since a first use of the handle apparatus or a reload assembly elapsing, and/or the like. For example, use of the manual retraction override of the handle apparatus can indicate that the handle apparatus or an attached reload assembly malfunctioned during use, requiring the manual retraction override to return the handle apparatus and/or the reload assembly to an initialized or “homed” state. As another example, the handle apparatus and/or a reload assembly can be enabled for use in a preset number (e.g., one, two, or more) of power cycles and/or firings before the handle apparatus and/or the reload assembly must be retired (e.g., disposed of, re-sterilized/reset). Thus, the error or fail state encountered at block 1402 can be the handle apparatus and/or the reload assembly being used for the maximum number of power cycles and/or firings. As still another example, the handle apparatus and/or a reload assembly can be enabled for use for a preset amount of time (e.g., 1 hours, 3 hours, 6 hours, 12 hours, 18 hours, 24 hours, etc.) following a first powerup and/or use of the handle apparatus and/or reload assembly. This can encourage use of a new/sterilized handle apparatus and/or reload assembly after the preset amount of time has elapsed since the first powerup and/or use. Thus, the error or fail state can be the preset amount of time elapsing.

At block 1404, the method 1400 continues by storing, in persistent memory of the handle apparatus, an error code associated with or corresponding to the error or fail state encountered and identified at block 1402. Thus, the error code can be retained and detected/read across multiple power cycles. In some embodiments, the method 1400 can include, at block 1404, communicating an indication of the error or fail state (e.g., an error code corresponding to the error or fail state) to the control unit, such as for storage on memory of the control unit and/or communication to a user/operator. At block 1406, the method 1400 continues by powering down the handle apparatus, thereby ending the power cycle during which (a) the error or fail state was encountered and (b) the corresponding error code was stored in persistent memory.

At block 1408, the method 1400 continues by receiving an indication of connection to power (e.g., to the power source 150 of FIG. 1, to the power source 250 of FIG. 2, to an internal power source such as a battery), an indication of powerup of the handle apparatus, and/or an indication of a connection (e.g., wired or wireless) to a control unit. Thus, in some embodiments, the handle apparatus can enter a subsequent (e.g., second) power cycle that is different from the power cycle described above during which the error or fail state was encountered and the corresponding error code was stored in persistent memory.

At blocks 1410 and 1412, the method 1400 continues by checking (e.g., scanning, reading) the persistent memory and determining whether there are one or more error codes stored to the persistent memory. As previously mentioned, because the persistent memory can retain error codes across multiple power cycles, error codes from one or more previous power cycles can be detected at blocks 1410 and 1412. The absence of error codes stored in the persistent memory can indicate that no error events or issues corresponding to the handle apparatus have been identified during previous power cycles of the handle apparatus, or at least that any error events or issues corresponding to the handle apparatus have been resolved and/or cleared during a previous power cycle. Thus, in the event that there are no error codes stored to the persistent memory (block 1412: No error code), the method 1400 continues to block 1414 by allowing the handle apparatus to proceed (e.g., to a next safety check and/or to an enabled state for use in a medical procedure).

On the other hand, in the event that one or more error codes are identified as being stored to persistent memory of the handle apparatus (block 1412: Error code exists), the method 1400 continues to block 1416. At block 1416, the method 1400 continues by determining whether the one or more error codes stored to the persistent memory of the handle apparatus correspond to error conditions/states and/or issues that are resolvable. In some embodiments, determining whether the one or more error codes stored to the persistent memory of the handle apparatus correspond to errors that are resolvable includes cross-referencing the one or more error codes against a lookup table of error codes to determine a type and resolvability associated with each of the one or more error codes. In the event that (e.g., all of) the error codes are resolvable (block 1416: Resolvable error code), the method 1400 continues to block 1418 attempting to resolve the corresponding error(s), such as by directing a user/operator (e.g., the physician) to resolve or reset the error(s). For example, in the event that an error code stored to the persistent memory of the handle apparatus relates to an error/issue with a particular reloadable cartridge, the method 1400 can include directing the user to disconnect the problematic reloadable cartridge assembly and reconnect another reloadable cartridge assembly to resolve/reset the error. In some embodiments, resolving/resetting an error can include erasing an associated error code stored in the persistent memory (e.g., by overwriting the associated error code with null data) and/or storing a flag in the persistent memory that a particular instance of the error code has been resolved/reset.

In the event that all or a subset of the error codes read from persistent memory and identified at blocks 1410 and 1412 is/are determined to be irresolvable (block 1420: Irresolvable error code), the method 1400 continues to block 1420 by directing the user to discard the handle apparatus. For example, if the manual retraction override of the handle apparatus was used during a previous power cycle, if the handle apparatus has reached a maximum allowable number of uses/firings, and/or if the present maximum amount of time since first powerup/use has elapsed, the method 1400 can include determining that the handle apparatus and/or a corresponding reload assembly is/are no longer suitable for use and must be discarded, re-sterilized, and/or reset (e.g., reloaded with staples). In some embodiments, the determination of whether (a) no error codes were identified in the persistent memory, (b) resolvable error codes were identified in the persistent memory, and/or (c) irresolvable error codes were identified in the persistent memory, can be communicated to a physician (or other user/operator) via LED lights on the handle apparatus and/or the control unit, a screen on the handle apparatus and/or the control unit, an external display, and/or the like. Also, in the event that one or more error codes are identified in the persistent memory, the handle apparatus and/or the control unit can prevent or otherwise limit powered and/or manual actuation of the handle apparatus and/or of a corresponding reload assembly.

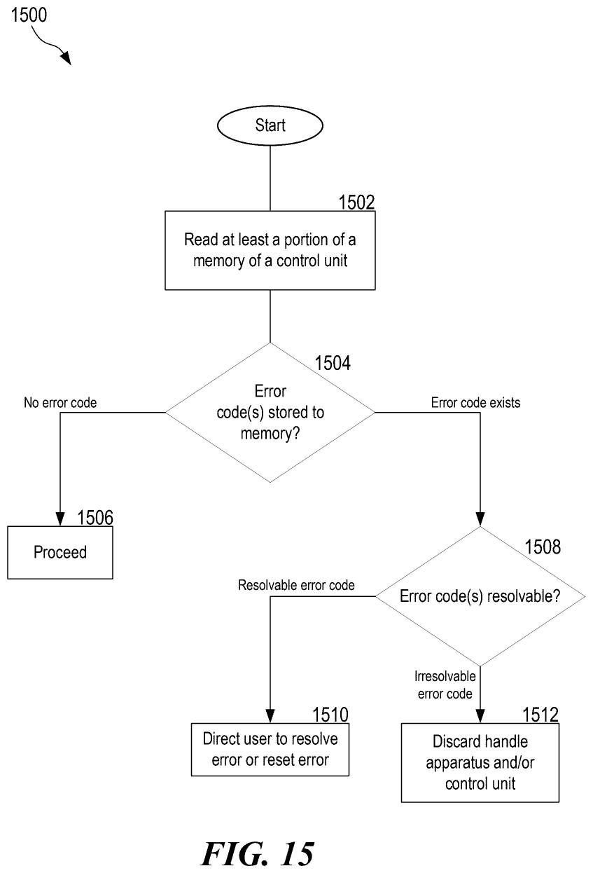

FIG. 15 is a flowchart illustrating a method 1500 for performing a control unit error code check in accordance with various embodiments of the present technology. In some embodiments, processors of a control unit (e.g., the processors 142 of the control unit 140 of FIG. 1) can execute the method 1500 to check for and respond to any error codes associated with a handle apparatus connected to the control unit and/or with the control unit itself. For example, as discussed above, when the handle apparatus encounters error or fail states, the handle apparatus can store an associated error code in persistent memory of the handle apparatus and/or transmit an indication of the error or fail state (e.g., the associated error code) to the control unit, such as for storage in memory of the control unit and/or communication to a user/operator. Thus, error codes stored in memory of the control unit can be associated with (a) error or fail states encountered by the control unit (e.g., during pervious power cycles of the control unit) and/or (b) error or fail state encountered by a handle apparatus associated with/connected to the control unit. The method 1500 begins at block 1502 by reading (e.g., scanning, checking) at least a portion of a memory of the control unit.

At block 1504, the method 1500 continues by determining whether any error codes are stored to the memory of the control unit. In the event that there are no error codes stored to the memory of the control unit (block 1504: No error code), the method 1500 continues to block 1506 by allowing the handle apparatus and/or the control unit to proceed (e.g., to a next safety check and/or to an enabled state for use in a medical procedure). On the other hand, in the event that there are one or more error codes stored to the memory of the control unit (block 1504: Error code exists), the method 1500 continues to block 1508 by identifying one or more errors associated with one or more of the error codes stored to the memory, and determining whether the one or more identified errors are resolvable. In some embodiments, identifying the one or more errors associated with the one or more error codes includes cross-referencing all or a subset of the one or more error codes against a lookup table to determine a type and resolvability of errors associated with all of the subset of the one or more error codes.

In the event that (e.g., all of) the error(s) is/are resolvable (block 1508: Resolvable error code), the method 1500 continues to block 1510 by attempting to resolve the corresponding error(s), such as by directing a user/operator (e.g., the physician) to resolve or reset the error(s). In some embodiments, resolving/resetting an error can include erasing an associated error code stored in the memory of the control unit (e.g., by overwriting the associated error code with null data) and/or storing a flag in the persistent memory that a particular instance of the error code has been resolved/reset.

In the event that all or a subset of the errors is/are determined to be irresolvable (block 1508: Irresolvable error code), the method 1500 continues to block 1512 by directing the user to discard the handle apparatus and/or the control unit. For example, if the manual retraction override of the handle apparatus was used, if the handle apparatus has reached a maximum allowable number of uses/firings, and/or if the present maximum amount of time since first powerup/use has elapsed, the method 1500 can include determining that the handle apparatus and/or a corresponding reload assembly is/are no longer suitable for use and must be discarded, re-sterilized, and/or reset (e.g., reloaded with staples). In some embodiments, the determination of whether (a) no error codes were identified in the memory of the control unit, (b) resolvable error codes were identified in the memory of the control unit, and/or (c) irresolvable error codes were identified in the memory of the control unit, can be communicated to the physician via LED lights on the handle apparatus and/or the control unit, a screen on the handle apparatus and/or the control unit, an external display, and/or the like. Also, in the event that one or more error codes are identified in the memory of the control unit, the handle apparatus and/or the control unit can prevent or otherwise limit powered and/or manual actuation of the handle apparatus and/or of a corresponding reload assembly.

### 4. Communication of System Operating State(s)

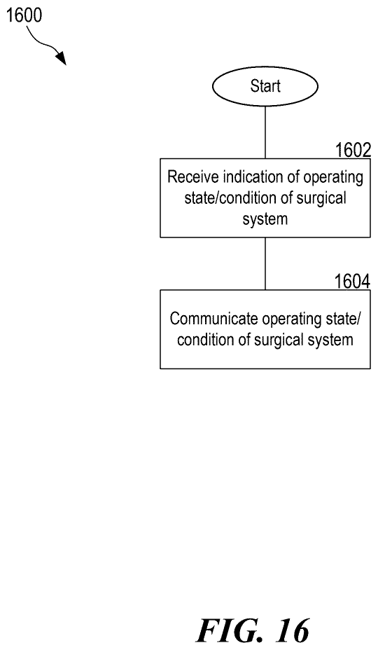

FIG. 16 is a flowchart illustrating a method 1600 for communicating a system operating state in accordance with various embodiments of the present technology. In some embodiments, processors of a handle apparatus (e.g., the processors 132 of the handle apparatus 130 of FIG. 1) and/or processors of a control unit (e.g., the processors 142 of the control unit 140 of FIG. 1) can execute the method 1600. The method 1600 begins at block 1602 by receiving an indication of an operating state or condition of a surgical system. The operating state or condition of the surgical system can be a condition encountered by a control unit, a handle apparatus, and/or a reload assembly connected to the handle apparatus. For example, the operating state or condition can be a normal or expected operating state or condition, and the indication received at block 1602 can be an indication that the system (or one of its subcomponents) is operating normally and/or as expected, such as without issues. As another example, the operating state or condition can be an operating state or condition encountered by the handle apparatus or a reload assembly attached to the handle apparatus. Examples of operating states or conditions encountered by the handle apparatus/reload assembly include a spike in current consumption of a motor of the handle assembly indicating (i) that a reload lockout mechanism was previously fired/engaged and is now preventing further distal movement of a drive assembly/blade assembly; (ii) that the reload assembly and handle apparatus are attempting to clamp, cut, and/or staple tissue that is too thick for the particular reload assembly installed on the handle apparatus; and/or (iii) that a blade assembly has reached the end of its cutting and/or stapling stroke and therefore is abutting against an end portion of the reload assembly. Other examples of operating states of conditions encountered by the handle apparatus/reload assembly include (i) the handle assembly/reload assembly currently in a lockout state (e.g., the reload assembly was previously fired, then retracted, and now attempting to be move distally without the reload assembly being detached and swapped out for another new/sterile/loaded reload assembly); (ii) a blade assembly reaching an end of stroke position (e.g., as determined using motor encoder readings). When the operating state or condition is an operating state or condition encountered by the handle apparatus or a reload assembly, the handle apparatus or a reload assembly can transmit an indication of the operating state or condition to the control unit such that the control unit receives the indication at block 1602 of the method. Additionally, or alternatively, the operating state or condition can be an operating state or condition encountered by the control unit, and the control unit can transmit an indication of the operating state or condition to the handle apparatus such that the handle apparatus receives the indication at block 1602 of the method.

At block 1604, the method 1600 continues by communicating the operating state or condition of the surgical system. In some embodiments, communicating the operating state or condition of the surgical system can include identifying the operating state or condition. For example, receiving the indication at block 1602 can include receiving a code corresponding to the operating state or condition. Continuing with this example, identifying the operating state or condition of the surgical system at block 1604 can include cross-referencing the code against entries of a lookup table that associates a plurality of codes with a corresponding plurality of operating states or conditions. In these and other embodiments, communicating the operating state or condition can include communicating the operating state or condition to a physician (or other user/operator) via LED lights on the handle apparatus and/or the control unit, a screen on the handle apparatus and/or the control unit, an external display, and/or the like. Additionally, or alternatively, communicating the operating state or condition can include mirror a communication presented by another component of the surgical system. For example, in the event that the operating state or condition is an operating state or condition encountered by the handle apparatus, the handle apparatus can communicate an indication of the operating state or condition (i) to the control unit (e.g., by transmitting the control unit a corresponding code) and (ii) to an operator (e.g., using LED lights and various visual indicators, such as one or more colors, flash sequences, etc.). Continuing with this example, the control unit can receive the indication of the operating state or condition from the handle apparatus, and can communicate its own indication of the operating state or condition to an operator (e.g., using LED lights and the same or different visual indicators; using a screen or display and presenting graphics, icons, text, etc. thereon; using audio or tactile feedback; etc.). Thus, the control unit in some embodiments can, at the control unit, mirror communication of the operating state or condition encountered by the handle apparatus.

As another example, in the event that the operating state or condition is an operating state or condition encountered by the control unit, the control unit can communicate an indication of the operating state or condition (i) to the handle apparatus and/or another computing device in communication with the control unit (e.g., by transmitting the handle apparatus and/or the other computing device a corresponding code) and (ii) to an operator (e.g., using LED lights and various visual indicators, such as one or more colors, flash sequences, etc.; using a graphics and/or text on a screen of the control unit; using auditory and/or haptic feedback; etc.). Continuing with this example, the handle apparatus and/or the other computing device can receive the indication of the operating state or condition from the control unit, and can communicate its own indication of the operating state or condition to an operator (e.g., using LED lights and the same or different visual indicators; using a screen or display and presenting graphics, icons, text, etc. thereon; using audio or tactile feedback; etc.). Thus, the handle apparatus and/or the other computing device in some embodiments, can, at the handle apparatus and/or the other computing device, mirror communication of the operating state or condition encountered by the control unit.

Referring to FIGS. 5-16 together, if one or more of the safety check methods and/or communication methods described herein results in identification of one or more errors and/or operating states, in some embodiments, the presence and the nature of the error/operating state can be communicated to a physician via graphical user interfaces (GUIs) including one or more overlays. Additional details regarding such GUIs and overlays are provided in U.S. Provisional Patent Application No. 63/703,084, titled “DISPLAY SYSTEMS FOR SURGICAL DEVICES, AND ASSOCIATED DEVICES AND METHODS,” and filed on Oct. 3, 2024, the disclosure of which is incorporated by reference herein in its entirety.

### D. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

To the extent any material incorporated herein by reference conflicts with the present disclosure, the present disclosure controls. Where the context permits, singular or plural terms may also include the plural or singular term, respectively. For example, throughout this disclosure, the singular terms “a,” “an,” and “the” include plural referents unless the context clearly indicates otherwise. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Moreover, as used herein, the phrases “based on,” “depends on,” “as a result of,” and “in response to” shall not be construed as a reference to a closed set of conditions. For example, a step that is described as “based on condition A” may be based on both condition A and condition B without departing from the scope of the present disclosure. In other words, as used herein, the phrase “based on” shall be construed in the same manner as the phrase “based at least in part on” or the phrase “based at least partially on.”

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10 (e.g., any and all subranges having a minimum value of equal to or greater than 1 and a maximum value of equal to or less than 10, such as 5.5 to 10).

The disclosure set forth above is not to be interpreted as reflecting an intention that any claim or example requires more features than those expressly recited in that claim or example. Rather, as the preceding examples and the following claims reflect, inventive aspects lie in a combination of fewer than all features of any single foregoing disclosed embodiment. Thus, the preceding examples and the following claims are hereby expressly incorporated into the Detailed Description, with each claim standing on its own as a separate embodiment. This disclosure includes all permutations of the independent claims with their dependent claims.

I/We claim:

1. A surgical system, comprising:
a surgical handle apparatus;
a control unit configured to be coupled to the surgical handle apparatus via a wired connection,
wherein the surgical handle apparatus and/or the control unit is/are configured to perform two or more safety check operations in response, at least in part, to the surgical handle apparatus being connected to the control unit via the wired connection, wherein the two or more safety check operations include at least two of the following:
a data integrity check to determine whether data stored to a memory of the surgical handle apparatus and/or a memory of the control unit is faulty or outdated,
a handshake protocol to confirm that the surgical handle apparatus and the control unit can properly communicate with one another,
a voltage check to confirm that a voltage supplied to the surgical handle apparatus is within an appropriate range and/or whether the surgical handle apparatus is viable for use in a medical procedure,
a power negotiation to confirm an identity of the surgical handle apparatus when connected to the control unit,
a motor encoder functionality check to confirm whether one or more encoders included in the surgical handle apparatus are functioning properly,
a reload assembly check to confirm that the surgical handle apparatus is not connected a reload assembly when initially connected to power, initially connected to the control unit, and/or initially powered on, or
an error code check to check for and respond to any error codes retained by the surgical handle apparatus.

2. The surgical system of claim 1 wherein the surgical handle apparatus is a surgical stapler handle apparatus.

3. The surgical system of claim 1 wherein the two or more safety check operations include the data integrity check, and wherein, to perform the data integrity check, the surgical handle and/or the control unit is/are configured to:
calculate a first checksum value based at least in part on data stored to a memory of the surgical handle apparatus or the control unit;
retrieve a second checksum value stored to the memory;
compare the first checksum value to the second checksum value; and
either—
clear the data integrity check based at least in part on a determination that the first checksum value matches the second checksum value, or
communicate an error based at least in part on a determination that the first checksum value does not match the second checksum value.

4. The surgical system of claim 1 wherein the two or more safety check operations include the handshake protocol, and wherein, to perform the handshake protocol, one of the surgical handle or the control unit is configured to:
receive handshake data from another of the surgical handle apparatus or the control unit;
transmit, in response to the received handshake data, an acknowledgement signal to the another of the surgical handle apparatus or the control unit;
exchange communication parameters with the other of the surgical handle apparatus or the control unit, wherein the communication parameters include at least one of a data transfer rate, an encryption scheme, or an error-checking protocol; and
establish communication with the other of the surgical handle apparatus or the control unit based at least in part on the exchanged communication parameters.

5. The surgical system of claim 1 wherein the two or more safety check operations include the voltage check, and wherein, to perform the voltage check, the surgical handle apparatus and/or the control unit is/are configured to:
measure a voltage (i) received at the surgical handle apparatus or (ii) output from the control unit; and
compare the measured voltage to an acceptable voltage range; and
either—
clear the voltage check based at least in part on a determination that the measure voltage falls within the acceptable voltage range, or
communicate an error based at least in part on a determination that the measured voltage falls outside of the acceptable voltage range.

6. The surgical system of claim 1 wherein the two or more safety check operations include the power negotiation, and wherein, to perform the power negotiation, the control unit is configured to:
apply a voltage signal to the surgical handle apparatus;
measure a voltage drop across the surgical handle apparatus;
identify appropriate operating parameters and/or appropriate power parameters for the surgical handle apparatus based at least in part on the measured voltage drop; and
supply the appropriate operating parameters and/or the appropriate power parameters to the surgical handle apparatus.

7. The surgical system of claim 1 wherein the two or more safety check operations include the motor encoder functionality check, and wherein, to perform the motor encoder functionality check, the surgical handle apparatus and/or the control unit is/are configured to:
receive an encoder signal corresponding to a timing during which a motor of the surgical handle apparatus is operated in a known manner;
compare the received encoder signal to an expected encoder signal corresponding to the operation of the motor in the known manner; and
either—
clear the motor encoder functionality check based at least in part on a determination that the received encoder signal matches the expected encoder signal, or
communicate an error based at least in part on a determination that the received encoder signal does not match the expected encoder signal.

8. The surgical system of claim 1 wherein the two or more safety check operations include the reload assembly check, and wherein, to perform the reload assembly check, the surgical handle apparatus or the control unit is configured to:
receive a signal indicating whether a reload assembly is connected to the surgical handle apparatus at powerup of the surgical handle apparatus; and
either—
clear the reload assembly check based at least in part on a determination that the received signal indicates that a reload assembly is not connected to the surgical handle apparatus at the powerup of the surgical handle apparatus, or
communicate an error based at least in part on a determination that the received signal indicates that a reload assembly is connected to the surgical handle apparatus at the powerup of the surgical handle apparatus.

9. The surgical system of claim 1 wherein the two or more safety check operations include the error code check, and wherein, to perform the error code check, the surgical handle apparatus or the control unit is configured to:
identify one or more error codes stored to memory of the surgical handle apparatus or the control unit; and
either—
resolve the one or more error codes and clear the error code check based at least in part on a determination that the one or more error codes correspond to one or more errors that are resolvable, or
communicate an error based at least in part on a determination that an error code of the one or more error codes corresponds to an error that is not resolvable.

10. The surgical system of claim 9 wherein the two or more errors correspond to one or more of the following conditions:
use of a manual retraction override of the surgical handle apparatus;
reaching a predetermined maximum number of reload assembly firings for the surgical handle apparatus or a predetermined maximum number of power cycles for the surgical handle apparatus;
a predetermined maximum amount of time elapsing since first use of the surgical handle apparatus or since first use of a corresponding reload assembly; or
an error associated with a reload assembly attached to the surgical handle apparatus.

11. The surgical system of claim 1 wherein the control unit is further configured to, based at least in part on a safety check operation of the one or more safety check operations failing, (a) provide a first indication of the safety check operation failing and/or (b) cause an external display to provide a second indication of the safety check operation failing.

12. The surgical system of claim 1 wherein the surgical handle apparatus and/or the control unit is/are further configured to, based at least in part on a safety check operation of the one or more safety check operations failing, prevent or limit powered use of the handle apparatus.

13. A method for operating a surgical system, the method comprising:
performing, using a surgical handle apparatus and/or a control unit separate from the surgical handle apparatus, two or more safety check operations in response, at least in part, to the surgical handle apparatus being placed in communication with the control unit,
wherein the two or more safety check operations include at least two of:
a data integrity check to determine whether data stored to a memory of the surgical handle apparatus and/or a memory of the control unit is faulty or outdated,
a handshake protocol to confirm that the surgical handle apparatus and the control unit can properly communicate with one another,
a voltage check to confirm that a voltage supplied to the surgical handle apparatus is within an appropriate range and/or whether the surgical handle apparatus is viable for use in a medical procedure,
a power negotiation to confirm an identity of the surgical handle apparatus when connected to the control unit,
a motor encoder functionality check to confirm whether one or more encoders included in the surgical handle apparatus are functioning properly,
a reload assembly check to confirm that the surgical handle apparatus is not connected a reload assembly when initially connected to power, initially connected to the control unit, and/or initially powered on, or
an error code check to check for and respond to any error codes retained by the surgical handle apparatus.

14. The method of claim 13 wherein performing the two or more safety check operations comprises performing the data integrity check, and wherein performing the data integrity check includes:
calculating a first checksum value based at least in part on data stored to a memory of the surgical handle apparatus or the control unit;
retrieving a second checksum value stored to the memory; and
comparing the first checksum value to the second checksum value; and
either—
clearing the data integrity check based at least in part on a determination that the first checksum value matches the second checksum value, or
communicating an error based at least in part on a determination that the first checksum value does not match the second checksum value.

15. The method of claim 13 wherein performing the two or more safety check operations comprises performing the handshake protocol, and wherein performing the handshake protocol includes:
receiving handshake data from one of the surgical handle apparatus or the control unit;
computing a hash value based at least in part on the handshake data; and
comparing the computed hash value to a known hash value; and
either—
clearing the handshake protocol based at least in part on a determination that the computed hash value matches the known hash value, or
communicating an error based at least in part on a determination that the computed hash value does not match the known hash value.

16. The method of claim 13 wherein performing the two or more safety check operations comprises performing the voltage check, and wherein performing the voltage check includes:
measuring a voltage (i) received at the surgical handle apparatus or (ii) output from the control unit;
comparing the measured voltage to an acceptable voltage range; and
either—
clearing the voltage check based at least in part on a determination that the measure voltage falls within the acceptable voltage range, or
communicating an error based at least in part on a determination that the measured voltage falls outside of the acceptable voltage range.

17. The method of claim 13 wherein performing the two or more safety check operations comprises:
applying a voltage signal to the surgical handle apparatus;
measuring a voltage drop across the surgical handle apparatus;
identifying appropriate operating parameters and/or appropriate power parameters for the surgical handle apparatus based at least in part on the measured voltage drop; and
supplying the appropriate operating parameters and/or the appropriate power parameters to the surgical handle apparatus.

18. The method of claim 13 wherein performing the two or more safety check operations comprises performing the motor encoder functionality check, and wherein performing the motor encoder functionality check includes:
receiving an encoder signal corresponding to a timing during which a motor of the surgical handle apparatus is operated in a known manner;
comparing the received encoder signal to an expected encoder signal corresponding to operation of the motor in the known manner; and
either—
clearing the motor encoder functionality check based at least in part on a determination that the received encoder signal matches the expected encoder signal, or
communicating an error based at least in part on a determination that the received encoder signal does not match the expected encoder signal.

19. The method of claim 13 wherein performing the two or more safety check operations comprises performing the reload assembly check, and wherein performing the reload assembly check includes:
receiving a signal indicating whether a reload assembly is connected to the surgical handle apparatus at powerup of the surgical handle apparatus; and
either—
clearing the reload assembly check based at least in part on a determination that the received signal indicates that a reload assembly is not connected to the surgical handle apparatus at the powerup of the surgical handle apparatus, or
communicating an error based at least in part on a determination that the received signal indicates that a reload assembly is connected to the surgical handle apparatus at the powerup of the surgical handle apparatus.

20. The method of claim 13 wherein performing the two or more safety check operations comprises performing the error code check, and wherein performing the error code check includes:
identifying one or more error codes stored to memory, the one or more error codes relating to one or more error conditions encountered by the surgical handle apparatus or the control unit; and
either—
clearing the error code check based at least in part on a determination that the one or more error conditions are resolvable, or
communicating an error based at least in part on a determination that an error condition of the one or more error conditions is not resolvable.
